(19) United States
(12) Patent Application Publication
(10) Pub. No.: US 2024/0208949 A1
(43) Pub. Date: Jun. 27, 2024

(54) COMPOSITIONS, FORMULATIONS, AND METHODS FOR HAIR TREATMENT

(71) Applicant: Oddity Labs, LLC, New York, NY (US)

(72) Inventors: Avinash BOPPANA, Boston, MA (US); Kongyu ZHANG, Boston, MA (US); Evan ZHAO, Woburn, MA (US); Connor Wilson COLEY, Cambridge, MA (US); Enzo Corey BENFANTI, Somerville, MA (US); Erin HORGAN, Marblehead, MA (US)

(21) Appl. No.: 18/528,113

(22) Filed: Dec. 4, 2023

Related U.S. Application Data
(63) Continuation of application No. PCT/US2023/082287, filed on Dec. 4, 2023.
(60) Provisional application No. 63/430,172, filed on Dec. 5, 2022.

(51) Int. Cl. C07D 405/04; A61K 31/4196; A61P 17/14

(52) U.S. Cl. CPC C07D 405/04; A61K 31/4196; A61P 17/14

(57) ABSTRACT

Compositions and formulations for hair treatment are provided herein. Methods for hair treatment, such as methods for preventing or treating hair loss or hair thinning, are also provided herein. The methods and formulations may promote hair growth, hair restoration or hair thickening, or increase hair density or hair growth rate.

### CROSS REFERENCE

This application is a continuation of International Patent Application No. PCT/US2023/082287, filed Dec. 4, 2023, which claims the benefit of U.S. Provisional Patent Application No. 63/430,172, filed Dec. 5, 2022, which is incorporated by reference herein in its entirety.

### SUMMARY

Hair loss or thinning is a common problem which is, for example, naturally occurring or chemically promoted through the long-term use of certain chemicals (e.g., commercial products or therapeutic drugs). Often such hair loss or thinning is accompanied by lack of hair re-growth which causes partial or full baldness. While hair loss is often thought of as a man's problem, at least a third of women will experience thinning hair at some point in their lives.

The disclosure provides a composition comprising a compound having a structure of Formula (I):

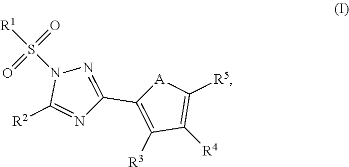

or a salt thereof,
wherein:
R¹ is selected from hydrogen, —OH, C₁₋₆ alkyl, and C₁₋₆ haloalkyl;
R² is selected from hydrogen, —OH, C₁₋₆ alkoxy, C₁₋₆ haloalkoxy, —SH, —S-alkyl, —S— haloalkyl, and —S—(CH₂CH₂—O)ₙ—R^{a}; wherein
n is an integer from 0 to 6, and
R^{a} is C₁₋₆ alkyl or C₁₋₆ haloalkyl;
A is NH, O or S; and
R³, R⁴, and R⁵ are independently selected from hydrogen, —OH, C₁₋₆ alkyl, and C₁₋₆ haloalkyl.

In some embodiments, W is C₁₋₆ alkyl. In some embodiments, the C₁₋₆ alkyl is selected from —CH₃, —(CH₂)₂CH₃, and —(CH₂)₄CH₃. In some embodiments, R² is —S-alkyl. In some embodiments, R² is —S—CH₃. In some embodiments, R² is —S—CH₂CH₃. In some embodiments, R² is —S—(CH₂CH₂—O)ₙ—R^{a}. In some embodiments, n is 1 or 2. In some embodiments, R^{a} is C₁₋₆ alkyl. In some embodiments, R^{a} is —CH₃. In some embodiments, A is O. In some embodiments, R³ is hydrogen. In some embodiments, R⁴ is hydrogen. In some embodiments, R⁵ is hydrogen.

In some embodiments, the compound of Formula (I) is selected from:

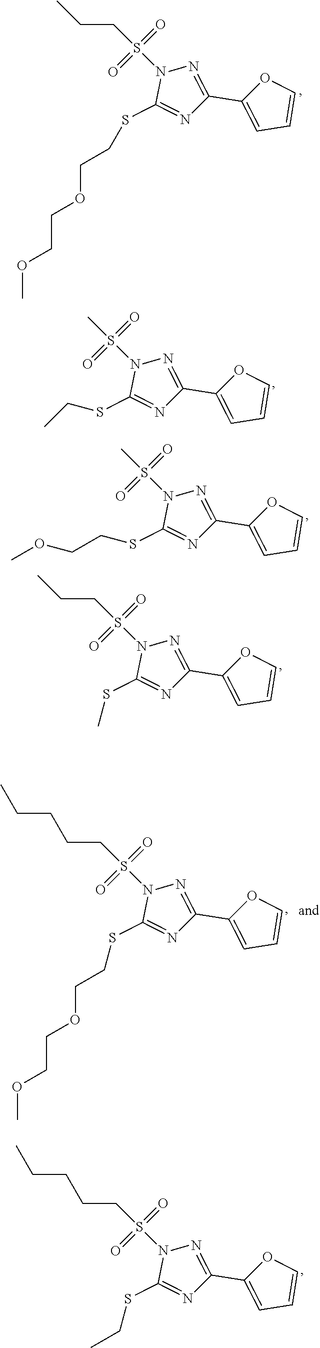

or a salt thereof. In some embodiments, the compound of Formula (I) is

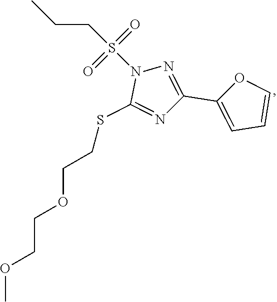 [SMILES: CCCS(=O)(=O)n1nc(-c2ccco2)nc1SCCOCCOC]

or a salt thereof.

In some embodiments, a weight % of the compound in the composition ranges from about 0.0001% to about 10% by weight relative to the total weight of the composition. In some embodiments, a weight % of the compound in the composition ranges from about 0.005% to about 10.0% by weight relative to the total weight of the composition. In some embodiments, a weight % of the compound in the composition ranges from about 0.01% to about 5% by weight relative to the total weight of the composition. In some embodiments, a weight % of the compound in the composition ranges from about 0.01% to about 2.0% by weight relative to the total weight of the composition.

In some embodiments, the composition is a pharmaceutical composition. In some embodiments, the composition further comprises at least one additive selected from the group consisting of pharmaceutically acceptable carriers, excipients, adjuvants, and diluents. In some embodiments, the pharmaceutical composition is formulated as a topically administrable composition. In some embodiments, the topically administrable composition comprises a solution, a suspension, an ointment, a paste, a lotion, a cream, a gel, a balm, or a medicated stick.

In some embodiments, the composition is a cosmetic composition. In some embodiments, the composition further comprises at least one additive selected from the group consisting of cosmetically acceptable carriers, excipients, adjuvants, and diluents. In some embodiments, the cosmetic composition is formulated as a toner, an emulsion, a cream, a gel, a shampoo, a soap, a serum, a spray, an oil, an ointment, a paste, a lotion, a balm, or a suspension.

In some embodiments, the composition provides pH of from about 2 to about 6. In some embodiments, the composition provides pH of from about 4 to about 5.

In some embodiments, the composition preserves the chemical structure and prevents hydrolysis/degradation of the compound of Formula (I).

In some embodiments, the composition is used for treating hair loss or hair thinning of a subject in need thereof. In some embodiments, the hair loss is selected from androgenic alopecia, alopecia areata, androgenetic alopecia, gynecologic alopecia, postpartum alopecia, seborrheic alopecia, non-rigid alopecia, senile alopecia, chemotherapy-induced alopecia, radiation-induced alopecia, male-pattern baldness, female-pattern baldness, cicatricial alopecia, alopecia areata telogen effluvium, traction alopecia, anagen effluvium, and combinations thereof. In some embodiments, the composition promotes hair strength, hair growth, hair restoration, hair thickening, or combinations thereof of the subject. In some embodiments, the composition increases hair density, hair growth rate, or combination thereof of the subject. In some embodiments, the hair is scalp hair, eyelash hair, eyebrow hair, facial hair, or combinations thereof.

This disclosure provides a method for treating hair loss or hair thinning. In some embodiments, the method comprises administering to a subject in need thereof a composition comprising a compound having a structure of Formula (I):

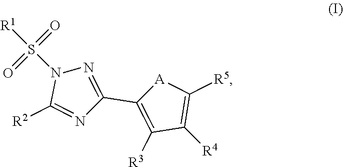

or a salt thereof,
wherein:
R¹ is selected from hydrogen, —OH, C₁₋₆ alkyl, and C₁₋₆ haloalkyl;
R² is selected from hydrogen, —OH, C₁₋₆ alkoxy, C₁₋₆ haloalkoxy, —SH, —S-alkyl, —S— haloalkyl, and —S—(CH₂CH₂—O)ₙ—R^{a}; wherein
n is an integer from 0 to 6, and
R^{a} is C₁₋₆ alkyl or C₁₋₆ haloalkyl;
A is NH, O or S; and
R³, R⁴, and R⁵ are independently selected from hydrogen, —OH, C₁₋₆ alkyl, and C₁₋₆ haloalkyl.

In some embodiments, R¹ is C₁₋₆ alkyl. In some embodiments, the C₁₋₆ alkyl is selected from —CH₃, —(CH₂)₂CH₃, and —(CH₂)₄CH₃. In some embodiments, R² is —S-alkyl. In some embodiments, R² is —S—CH₃. In some embodiments, R² is —S—CH₂CH₃. In some embodiments, R² is —S—(CH₂CH₂-O)ₙ—R^{a}. In some embodiments, n is 1 or 2. In some embodiments, R^{a} is C₁₋₆ alkyl. In some embodiments, R^{a} is —CH₃. In some embodiments, A is O. In some embodiments, R³ is hydrogen. In some embodiments, R⁴ is hydrogen. In some embodiments, R⁵ is hydrogen.

In some embodiments, the compound of Formula (I) is selected from:

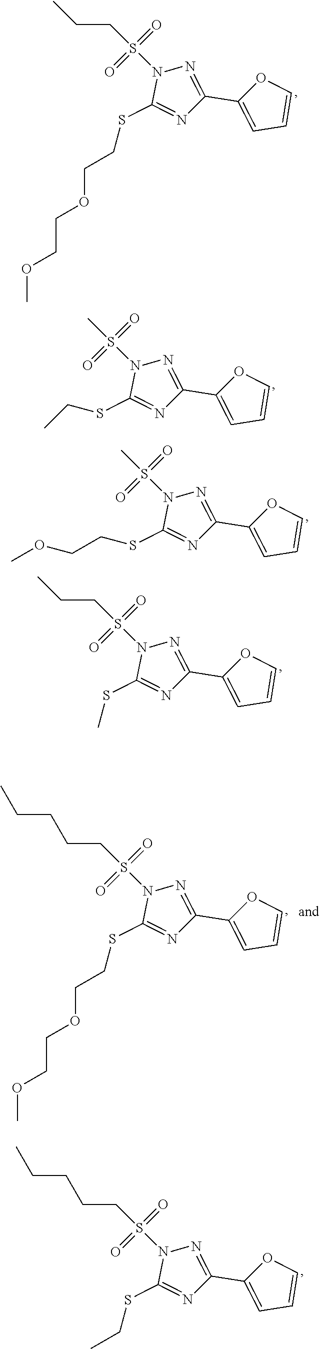

or a salt thereof. In some embodiments, the compound of Formula (I) is

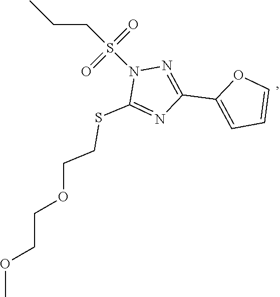 [SMILES: CCCS(=O)(=O)n1nc(-c2ccco2)nc1SCCOCCOC]

or a salt thereof.

In some embodiments, a weight % of the compound in the composition ranges from about 0.0001% to about 10% by weight relative to the total weight of the composition. In some embodiments, a weight % of the compound in the composition ranges from about 0.005% to about 10.0% by weight relative to the total weight of the composition. In some embodiments, a weight % of the compound in the composition ranges from about 0.01% to about 5% by weight relative to the total weight of the composition. In some embodiments, a weight % of the compound in the composition ranges from about 0.01% to about 2.0% by weight relative to the total weight of the composition.

In some embodiments, the composition is used for preventing or treating hair loss or hair thinning of a subject in need thereof, wherein the subject has been diagnosed with hair loss or hair thinning. In some embodiments, the composition is used for preventing or treating hair loss or hair thinning of a subject in need thereof, wherein the subject has not been diagnosed with hair loss or hair thinning.

In some embodiments, the composition is a pharmaceutical composition. In some embodiments, the pharmaceutical composition further comprises at least one additive selected from the group consisting of pharmaceutically acceptable carriers, excipients, adjuvants, and diluents. In some embodiments, the pharmaceutical composition is formulated into a variety of topically administrable compositions, including but are not limited to a solution, a suspension, an ointment, a paste, a lotion, a cream, a gel, a balm, and a medicated stick.

In some embodiments, the pharmaceutical composition is administered one to three times a day. In some embodiments, the pharmaceutical composition is administered two times a day. In some embodiments, the pharmaceutical composition is administered three times a day. In some embodiments, the pharmaceutical composition is administered for at least five consecutive days. In some embodiments, the pharmaceutical composition is administered for at least seven consecutive days. In some embodiments, the pharmaceutical composition is administered at least for about 15 days, about 30 days, about 60 days, about 90 days, about 6 months, or a year.

In some embodiments, the composition is a cosmetic composition. In some embodiments, the cosmetic composition further comprises at least one additive selected from the group consisting of cosmetically acceptable carriers, excipients, adjuvants, and diluents. In some embodiments, the cosmetic composition is formulated as a toner, an emulsion, a cream, a gel, a shampoo, a soap, a serum, a spray, or an oil.

In some embodiments, the hair loss is selected from androgenic alopecia, alopecia areata, androgenetic alopecia, gynecologic alopecia, postpartum alopecia, seborrheic alopecia, non-rigid alopecia, senile alopecia, chemotherapy-induced alopecia, radiation-induced alopecia, male-pattern baldness, female-pattern baldness, cicatricial alopecia, alopecia areata telogen effluvium, traction alopecia, anagen effluvium, and combinations thereof. In some embodiments, the method promotes hair strength, hair growth, hair restoration, hair thickening, or combinations thereof of the subject. In some embodiments, the method increases hair density, hair growth rate, or combination thereof of the subject. In some embodiments, the hair is scalp hair, eyelash hair, eyebrow hair, or facial hair.

This disclosure also provides a kit comprising the composition disclosed herein. In some embodiments, the kit further comprises an applicator. In some embodiments, the applicator comprises a brush, a comb, a cotton swab, a spatula, a spoon, a dropper, a spray nozzle, a squeeze bottle, a tottle, or a combination thereof. In some embodiments, the kit further comprises written instructions for using the composition in a hair treatment.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings (also “Figure” and “FIG.” herein), of which:

FIGS. 1A and 1B illustrate the activity of example compounds, in accordance with one or more embodiments of the present disclosure. FIG. 1A shows the percentage increase in daily proliferation of human follicle dermal papilla cells, while FIG. 1B displays normalized relative proliferation of human follicle dermal papilla cells after six hours of four consecutive days of heat treatment at 60° C. Data represents mean±standard error of the mean (s.e.m) for cells derived from three donors, with ages ranging from 25-83 years old. The presented data is representative of at least three experimental replicates. FIGS. 2A and 2B show LC-UV chromatograms of compound I-1, in accordance with one or more embodiments of the present disclosure. FIG. 2A displays LC-UV chromatogram of compound I-1 in the ethyl acetate (EtOAc)/heptane 1:1 solvent system, while FIG. 2B displays LC-UV chromatogram of compound I-1 in the 0.1% (v/v) formic acid (HCOOH) in water (H₂O)/0.1% (v/v) HCOOH in acetonitrile (MeCN) 1:1 solvent system.

FIGS. 3A, 3B, 3C, and 3D show safety profiles of compounds I-1 to I-5, in accordance with one or more embodiments of the present disclosure. FIG. 3A presents normalized β-galactosidase activity (quantified via absorbance) from the SOS chromotest using a known genotoxin (4NQ; 4-nitroquinoline-1-oxide) and example compounds at various concentrations. The SOS chromotest is a well-established bacteria-based test for genotoxicity. FIG. 3B illustrates normalized caspase 3/7 activity in HepG2 cells, an immortalized liver cell line commonly used to study apoptosis induced by small molecules. RX503 and Curcumin were included as known activators of caspase-3/7. FIG. 3C is normalized relative proliferation of compound I-1 cultured with human primary fibroblasts, keratinocytes, endothelial cells, melanocytes, and skeletal muscle cells. The Nrf2-ARE pathway is a master regulator of cytoprotective responses to oxidative stress, and serves as an early indicator of skin sensitization. Nrf2 (nuclear factor erythroid 2-related factor) is a transcription factor that binds to antioxidant responsive elements (ARE). By fusing ARE to the light-producing luciferase gene, a KeratinoSens Nrf2-ARE reporter assay was constructed, whereby the luciferase signal directly correlates to Nrf2-ARE pathway activation. FIG. 3D is normalized ARE-luciferase activity using known sensitizing compounds (fisetin and RH275) and example compounds. Cells were treated with either the example compounds or vehicle control containing correspondingly % v/v-matched amounts of DMSO spanning a >1-log concentration range. Data represents mean±standard deviation (s.d.) and is representative of at least two experimental replicates.

FIG. 4A shows a schematic representation of model system of culturing Caco-2 cells for biological assay, in accordance with one or more embodiments of the present disclosure.

FIG. 4B shows apparent permeability as measured by raw luminescence signal of a Caco-2 monolayer dosed with DMSO or example compounds, in accordance with one or more embodiments of the present disclosure.

FIG. 5 shows CYP3A4 inhibition activity of example compounds at various concentrations, in accordance with one or more embodiments of the present disclosure.

FIG. 6 shows generation of intracellular ROS of example compounds at various concentrations, in accordance with one or more embodiments of the present disclosure.

FIGS. 7A, 7B, 7C, and 7D show HPLC-UV analysis of compound I-1, in accordance with one or more embodiments of the present disclosure. FIG. 7A displays the UV-traces of the freshly synthesized material post-workup at 215 nm and FIG. 7B exhibits the UV-traces of the freshly synthesized material post-workup at 254 nm. FIG. 7C presents the UV-traces of the material after 24 hours of open-air exposure at 215 nm, and FIG. 7D displays the UV-traces of the material after 24 hours of open-air exposure at 254 nm.

FIGS. 8A, 8B, 8C, and 8D show HPLC-UV analysis of compound I-1 in a solution containing 1,3-Dimethyl-2-imidazolidinone (DMI) after exposure to air, in accordance with one or more embodiments of the present disclosure. FIG. 8A displays the UV-traces of the closed-air experiment at 215 nm, and FIG. 8B exhibits the UV-traces of the closed-air experiment at 254 nm. FIG. 8C presents the UV-traces of the open-air experiment at 215 nm, and FIG. 8D shows the UV-traces of the open-air experiment at 254 nm.

FIGS. 9A and 9B show NMR results of compound I-1 after distillation at 160° C. (FIG. 9A) and 185° C. (FIG. 9B), in accordance with one or more embodiments of the present disclosure.

FIG. 10 shows HPLC-UV analysis shows minimal hydrolysis of I-1 after 8 months at room temperature, in accordance with one or more embodiments of the present disclosure.

FIG. 11 shows normalized relative proliferation of human follicle dermal papilla cells using compound I-1 in combination with compound VII-3 at various concentration ratios, in accordance with one or more embodiments of the present disclosure.

### DETAILED DESCRIPTION OF THE INVENTION

Disclosed herein are compounds useful for treating hair loss or hair thinning of a subject in need thereof. In certain aspects, the disclosure provides methods for preventing or treating hair loss or hair thinning (whether or not a diagnosis of hair loss or hair thinning has been made).

### DEFINITIONS

Unless defined otherwise, all technical and scientific terms used herein have the same meaning as is commonly understood by one of skill in the art to which this invention belongs.

As used in the specification and claims, the singular form “a”, “an” and “the” includes plural references unless the context clearly dictates otherwise.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

The term “cycloalkyl” refers to a saturated ring in which each atom of the ring is carbon. Cycloalkyl may include monocyclic and polycyclic rings such as 3- to 10-membered monocyclic rings, 5- to 12-membered bicyclic rings, spiro bicycles, and 5- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally further substituted as described herein.

The term “heterocycle” as used herein refers to a saturated, unsaturated or aromatic ring comprising one or more heteroatoms. Example heteroatoms include N, O, Si, P, B, and S atoms. Heterocycles include 3- to 10-membered monocyclic rings, 6- to 12-membered bicyclic rings, 5- to 12-membered spiro bicycles, and 5- to 12-membered bridged rings. A bicyclic heterocycle includes any combination of saturated, unsaturated and aromatic bicyclic rings, as valence permits. In an example embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. A bicyclic heterocycle further includes spiro bicylic rings e.g., 5 to 12-membered spiro bicycles.

“heteroaryl” or “aromatic heterocycle” refers to a radical derived from a heteroaromatic ring radical that comprises one to eleven carbon atoms and at least one heteroatom wherein each heteroatom may be selected from N, O, and S. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, pyridine, pyrimidine, oxazole, furan, pyran, thiophene, isoxazole, benzimidazole, benzthiazole, and imidazopyridine. An “X-membered heteroaryl” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazine (═N—NH₂), —R^{bb}—OR^{aa}, —R^{bb}—OC(O)—R^{aa}, —R^{bb}—OC(O)—OR^{aa}, —R^{bb}—OC(O)—N(R^{aa})₂, —R^{bb}—N(R^{aa})₂, —R^{bb}—C(O)R^{aa}, —R^{bb}—C(O)OR^{aa}, —R^{bb}—(O)N(R^{aa})₂, —R^{bb}—O—R^{cc}—C(O)N(R^{aa})₂, —R^{bb}—N(R^{aa})C(O)OR^{aa}, —R^{bb}—N(R^{aa})C(O)R^{aa}, —R^{bb}—N(R^{aa})S(O)ₜR^{aa} (where t is 1 or 2), —R^{bb}—S(O)ₜR^{aa} (where t is 1 or 2), —R^{bb}—S(O)ₜOR^{aa} (where t is 1 or 2), and —R^{bb}—S(O)ₜN(R^{aa})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{bb}—OR^{aa}, —R^{bb}—OC(O)—R^{aa}, —R^{bb}—OC(O)—OR^{aa}, —R^{bb}—OC(O)—N(R^{aa})₂, —R^{bb}—N(R^{aa})₂, —R^{bb}—C(O)R^{aa}, —R^{bb}—C(O)OR^{aa}, —R^{bb}—(O)N(R^{aa})₂, —R^{bb}—O—R^{cc}—C(O)N(R^{aa})₂, —R^{bb}—N(R^{aa})C(O)OR^{aa}, —R^{bb}—N(R^{aa})C(O)R^{aa}, —R^{bb}—N(R^{aa})S(O)ₜR^{aa} (where t is 1 or 2), —R^{bb}—S(O)ₜR^{aa} (where t is 1 or 2), —R^{bb}—S(O)ₜOR^{aa} (where t is 1 or 2), and R^{bb}—S(O)ₜN(R^{aa})₂ (where t is 1 or 2); wherein each R^{aa} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{aa}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{bb}—OR^{aa}, —R^{bb}—OC(O)—R^{aa}, —R^{bb}—OC(O)—OR^{aa}, —R^{bb}—OC(O)—N(R^{aa})₂, —R^{bb}—N(R^{aa})₂, —R^{bb}—C(O)R^{aa}, —R^{bb}—C(O)OR^{aa}, —R^{bb}—(O)N(R^{aa})₂, —R^{bb}—O—R^{cc}—C(O)N(R^{aa})₂, —R^{bb}—N(R^{aa})C(O)OR^{aa}, —R^{bb}—N(R^{aa})C(O)R^{aa}, —R^{bb}—N(R^{aa})S(O)ₜR^{aa} (where t is 1 or 2), —R^{bb}—S(O)ₜR^{aa} (where t is 1 or 2), —R^{bb}—S(O)ₜOR^{aa} (where t is 1 or 2), and R^{bb}—S(O)ₜN(R^{aa})₂ (where t is 1 or 2); and wherein each R^{bb} is independently selected from a direct bond or a straight or branched alkylene, alkenylene, or alkynylene chain, and each R^{cc} is a straight or branched alkylene, alkenylene or alkynylene chain.

Double bonds to oxygen atoms, such as oxo groups, are represented herein as both “═O” and “(O)”. Double bonds to nitrogen atoms are represented as both “═NR” and “(NR)”. Double bonds to sulfur atoms are represented as both “═S” and “(S)”.

The term “exemplary” as used herein means “serving as an example, instance, or illustration.” Any embodiment described herein as “exemplary” is not to be construed as preferred or advantageous over other embodiments.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier may be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

In certain embodiments, the term “cosmetically acceptable salt” means any salt that is cosmetically tolerated if used appropriately for a cosmetic treatment especially if used on or applied to humans and/or mammals. In certain embodiments, these salts include, but are not restricted to the salts used to form base addition salts, either inorganic, such as for example and in a non-limiting sense, lithium, sodium, potassium, calcium, magnesium or aluminum, among others, or organic such as for example and in a non-limiting sense, ethylamine, diethylamine, ethylenediamine, ethanolamine, diethanolamine, arginine, lysine, histidine, or piperazine among others; or acid addition salts, either organic, such as for example and in a non-limiting sense, acetate, citrate, lactate, malonate, maleate, tartrate, fumarate, benzoate, aspartate, glutamate, succinate, oleate, trifluoroacetate, oxalate, pamoate or gluconate among others, or inorganic, such as for example and in a non-limiting sense, chloride, sulfate, borate, or carbonate among others.

A “cosmetically effective amount” as used herein refers to the amount of a compound sufficient to improve the outward physical appearance of a subject. It is to be understood that a “cosmetically effective” amount can vary from subject to subject, due to numerous factors including for example age, weight, general condition of the subject, the condition being treated, the severity of the condition being treated, and the judgment of the prescribing physician.

The phrase “cosmetically acceptable excipient” or “cosmetically acceptable carrier” as used herein comprises as a pharmaceutical cream base, an oil-in-water emulsion, a water-in-oil emulsion, a gel, or the like. The skilled artisan will understand that the appropriate carriers typically will contain ingredients, such as those typically found in the cosmetic and cosmeceutical fields: oils, waxes or other standard fatty substances, or conventional gelling agents and/or thickeners; emulsifiers; moisturizing agents; emollients; sunscreens; hydrophilic or lipophilic active agents; agents for combatting free radicals; preservatives; basifying or acidifying agents; fragrances; surfactants; fillers; natural products or extracts of natural product, such as aloe or green tea extract; vitamins; or coloring materials.

The term “in vivo” generally refers to an event that takes place in a subject's body.

The term “in vitro” generally refers to an event that takes place outside of a subject's body. For example, an in vitro assay encompasses any assay run outside of a subject. In vitro assays encompass cell-based assays in which cells alive or dead are employed. In vitro assays also encompass a cell-free assay in which no intact cells are employed.

The term “optional” or “optionally” means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

The term “pharmaceutically acceptable carrier, diluent or excipient” includes without limitation any adjuvant, carrier, excipient, glidant, sweetening agent, diluent, preservative, dye, colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

### COMPOUNDS

The present disclosure provides compounds, salts thereof, and compositions, formulations, methods thereof, for treating hair loss or hair thinning of a subject in need thereof. The compounds or salts thereof may have a structural formula (I). The compounds or salts thereof may be selected from those forth in TABLE 1, or any subset thereof. The compounds and salts thereof disclosed herein may be used in method(s) of this disclosure.

In certain aspects, disclosed herein is a compound having a structure of Formula (I):

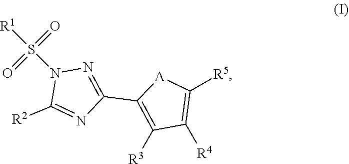

or a salt thereof,
wherein:
R¹ is selected from hydrogen, —OH, C₁₋₆ alkyl, and C₁₋₆ haloalkyl;
R² is selected from hydrogen, —OH, C₁₋₆ alkoxy, C₁₋₆ haloalkoxy, —SH, —S—C₁₋₆ alkyl, —S— haloalkyl, and —S—(CH₂CH₂—O)ₙ—R^{a}; wherein
n is an integer from 0 to 6, and
R^{a} is C₁₋₆ alkyl or C₁₋₆ haloalkyl;
A is NH, O or S; and
R³, R⁴, and R⁵ are independently selected from hydrogen, —OH, C₁₋₆ alkyl, and C₁₋₆ haloalkyl.

In some embodiments, R¹ is C₁₋₆ alkyl. In some embodiments, R¹ is hydrogen. In some embodiments, R¹ is —OH. In some embodiments, R¹ is C₁₋₆ haloalkyl. In some embodiments, R¹ is selected from —CH₃, —(CH₂)₂CH₃, and —(CH₂)₄CH₃. In some embodiments, R¹ is C₁ alkyl. In some embodiments, R¹ is —CH₃. In some embodiments, R¹ is C₂ alkyl. In some embodiments, R¹ is —CH₂CH₃. In some embodiments, R¹ is C₃ alkyl. In some embodiments, R¹ is —CH₂CH₂CH₃. In some embodiments, R¹ is C₄ alkyl. In some embodiments, R¹ is —(CH₂)₃CH₃. In some embodiments, R¹ is C₅ alkyl. In some embodiments, R¹ is —(CH₂)₄CH₃. In some embodiments, R¹ is C₆ alkyl. In some embodiments, R¹ is —(CH₂)₅CH₃. In some embodiments, R² is hydrogen. In some embodiments, R² is —OH. In some embodiments, R² is C₁₋₆ alkoxy. In some embodiments, R² is C₁₋₆ haloalkoxy. In some embodiments, R² is —SH. In some embodiments, R² is —S— C₁₋₆ alkyl. In some embodiments, R² is —S-haloalkyl. In some embodiments, R² is —S— (CH₂CH₂—O)ₙ—R^{a}. In some embodiments, n is 1 or 2. In some embodiments, R² is is —S—CH₃. In some embodiments, R² is —S—CH₂CH₃. In some embodiments, R² is —S—(CH₂)₂CH₃. In some embodiments, R² is —S—(CH₂)₃CH₃. In some embodiments, R² is —S—(CH₂)₄CH₃. In some embodiments, R² is —S—(CH₂)₅CH₃. In some embodiments, R² is —S—CH₂CH₂—O—R^{a}. In some embodiments, R² is —S—(CH₂CH₂—O)₂—R^{a}. In some embodiments, R^{a} is C₁₋₆ alkyl. In some embodiments, R^{a} is —CH₃. In some embodiments, R^{a} is C₂ alkyl. In some embodiments, R^{a} is C₃ alkyl. In some embodiments, R^{a} is C₄ alkyl. In some embodiments, R^{a} is C₅ alkyl. In some embodiments, R^{a} is C₆ alkyl. In some embodiments, R² is —S—CH₂CH₂—O—CH₃. In some embodiments, R² is —S—CH₂CH₂—O—CH₂CH₃. In some embodiments, R² is —S—CH₂CH₂—O—(CH₂)₂CH₃. In some embodiments, R² is —S—(CH₂CH₂—O)₂—CH₃. In some embodiments, R² is —S—(CH₂CH₂—O)₂—CH₂CH₃. In some embodiments, A is O. In some embodiments, A is NH. In some embodiments, A is S. In some embodiments, R³ is hydrogen. In some embodiments, R³ is —OH. In some embodiments, R³ is C₁₋₆ alkyl. In some embodiments, R³ is C₁₋₆ haloalkyl. In some embodiments, R⁴ is hydrogen. In some embodiments, R⁴ is —OH. In some embodiments, R⁴ is C₁₋₆ alkyl. In some embodiments, R⁴ is C₁₋₆ haloalkyl. In some embodiments, R⁵ is hydrogen. In some embodiments, R⁵ is —OH. In some embodiments, R⁵ is C₁₋₆ alkyl. In some embodiments, R⁵ is C₁₋₆ haloalkyl.

In some embodiments, the compound having structural Formula (I) is

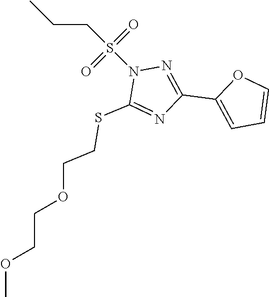 [SMILES: CCCS(=O)(=O)n1nc(-c2ccco2)nc1SCCOCCOC]

or a salt thereof. In some embodiments, the compound having structural Formula (I) is

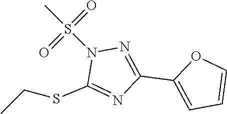 [SMILES: CCSc1nc(-c2ccco2)nn1S(C)(=O)=O]

or a salt thereof. In some embodiments, the compound having structural Formula (I) is

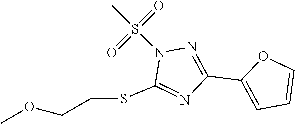 [SMILES: COCCSc1nc(-c2ccco2)nn1S(C)(=O)=O]

or a salt thereof. In some embodiments, the compound having structural Formula (I) is

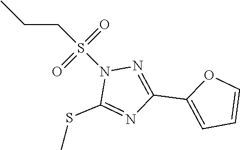 [SMILES: CCCS(=O)(=O)n1nc(-c2ccco2)nc1SC]

or a salt thereof. In some embodiments, the compound having structural Formula (I) is

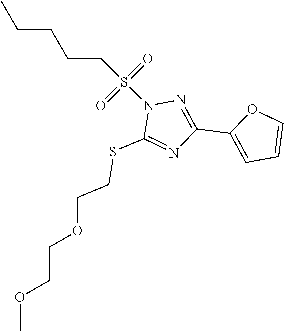 [SMILES: CCCCCS(=O)(=O)n1nc(-c2ccco2)nc1SCCOCCOC]

or a salt thereof. In some embodiments, the compound having structural Formula (I) is

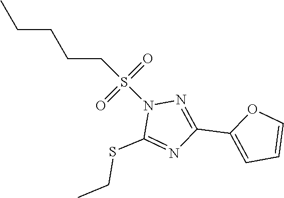 [SMILES: CCCCCS(=O)(=O)n1nc(-c2ccco2)nc1SCC]

or a salt thereof. In some embodiments, the compound having structural Formula (I) is

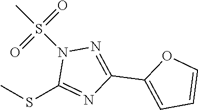 [SMILES: CSc1nc(-c2ccco2)nn1S(C)(=O)=O]

or a salt thereof.

In some embodiments, the compound having structural Formula (I) is selected from those set forth in TABLE 1, and salts thereof.

TABLE 1
Example Compounds of Formula (I)

| ID # | Chemical Structure |
|---|---|
| I-1 | 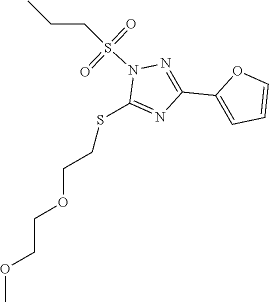 [SMILES: CCCS(=O)(=O)n1nc(-c2ccco2)nc1SCCOCCOC] |
| I-2 | 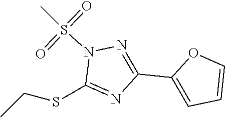 [SMILES: CCSc1nc(-c2ccco2)nn1S(C)(=O)=O] |
| I-3 | 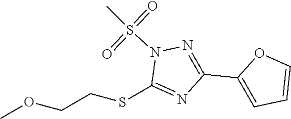 [SMILES: COCCSc1nc(-c2ccco2)nn1S(C)(=O)=O] |
| I-4 | 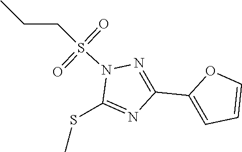 [SMILES: CCCS(=O)(=O)n1nc(-c2ccco2)nc1SC] |
| I-5 | 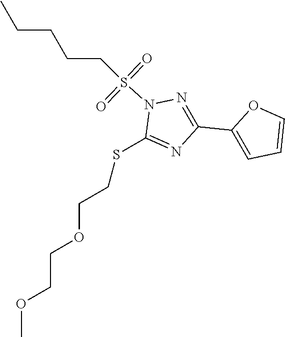 [SMILES: CCCCCS(=O)(=O)n1nc(-c2ccco2)nc1SCCOCCOC] |
| I-6 | 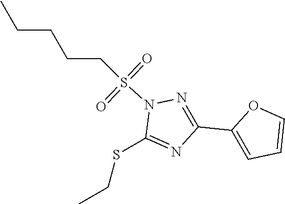 [SMILES: CCCCCS(=O)(=O)n1nc(-c2ccco2)nc1SCC] |
| VII-3 | 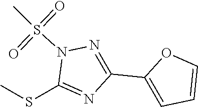 [SMILES: CSc1nc(-c2ccco2)nn1S(C)(=O)=O] |

For the synthesis methods of the compounds disclosed herein, see, e.g., WO2009031709 A1; Journal fuer Praktische Chemie (Leipzig), 323 (2), 3030310, 1981; European Journal of Medicinal Chemistry, 47, 138-142, 2012; Pharmaceutical Chemistry Journal, 41, 70-473, 2007; A.MA.J. Diseases Children, 97, 66-71, 1959; WO2003072099 A1; Chemiker-Zeitung, 111, 159-166, 1987; Journal of Heterocyclic Chemistry, 25(3), 959-968, 1988; and Russian Chemical Bulletin, 52(6), 1386-1398, 2003, each of which is incorporated herein by reference.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley and Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### COMPOSITIONS

Disclosed herein is a composition comprising a compound having a structure of Formula (I):

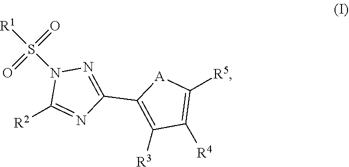

or a salt thereof,
wherein:
R¹ is selected from hydrogen, —OH, C₁₋₆ alkyl, and C₁₋₆ haloalkyl;
R² is selected from hydrogen, —OH, C₁₋₆ alkoxy, C₁₋₆ haloalkoxy, —SH, —S-alkyl, —S— haloalkyl, and —S—(CH₂CH₂—O)ₙR^{a}; wherein
n is an integer from 0 to 6, and
R^{a} is C₁₋₆ alkyl or C₁₋₆ haloalkyl;
A is NH, O or S; and
R³, R⁴, and R⁵ are independently selected from hydrogen, —OH, C₁₋₆ alkyl, and C₁₋₆ haloalkyl.

In some embodiments, R¹ is C₁₋₆ alkyl. In some embodiments, R¹ is selected from —CH₃, —(CH₂)₂CH₃, and —(CH₂)₄CH₃. In some embodiments, R¹ is —CH₃. In some embodiments, R¹ is —(CH₂)₂CH₃. In some embodiments, R¹ is —(CH₂)₄CH₃. In some embodiments, R² is —S-alkyl. In some embodiments, R² is —S—CH₃. In some embodiments, R² is —S—CH₂CH₃. In some embodiments, R² is —S—(CH₂CH₂-O)ₙ—R^{a}. In some embodiments, n is 1 or 2. In some embodiments, R^{a} is C₁₋₆ alkyl. In some embodiments, R^{a} is —CH₃. In some embodiments, R² is —S—CH₂CH₂—O—CH₃. In some embodiments, R² is —S—(CH₂CH₂-O)₂—CH₃. In some embodiments, A is O. In some embodiments, R³ is hydrogen. In some embodiments, R⁴ is hydrogen. In some embodiments, R⁵ is hydrogen.

In some embodiments, the compound of Formula (I) is selected from:

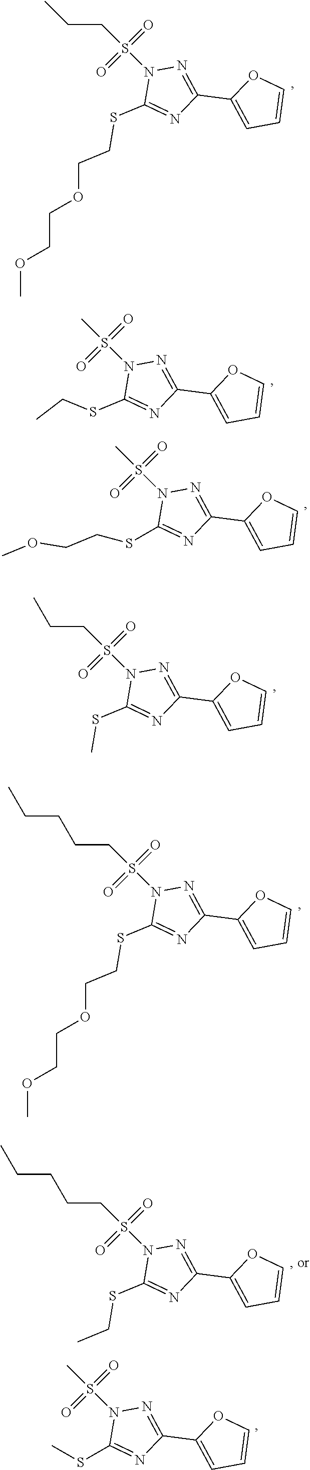

or a salt thereof.

In some embodiments, the compound of Formula (I) is

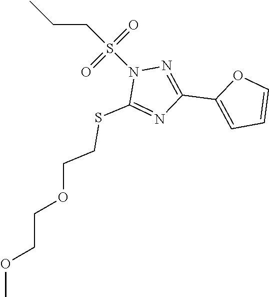 [SMILES: CCCS(=O)(=O)n1nc(-c2ccco2)nc1SCCOCCOC]

or a salt thereof. In some embodiments, the compound of Formula (I) is

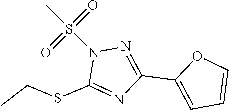 [SMILES: CCSc1nc(-c2ccco2)nn1S(C)(=O)=O]

or a salt thereof. In some embodiments, the compound of Formula (I) is

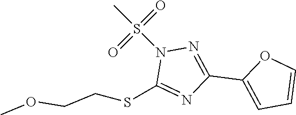 [SMILES: COCCSc1nc(-c2ccco2)nn1S(C)(=O)=O]

or a salt thereof. In some embodiments, the compound of Formula (I) is

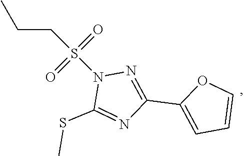 [SMILES: CCCS(=O)(=O)n1nc(-c2ccco2)nc1SC]

or a salt thereof. In some embodiments, the compound of Formula (I) is

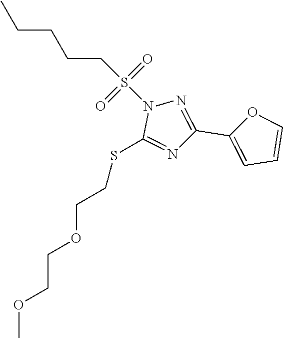 [SMILES: CCCCCS(=O)(=O)n1nc(-c2ccco2)nc1SCCOCCOC]

or a salt thereof. In some embodiments, the compound of Formula (I) is

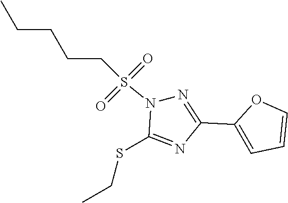 [SMILES: CCCCCS(=O)(=O)n1nc(-c2ccco2)nc1SCC]

or a salt thereof. In some embodiments, the compound of Formula (I) is

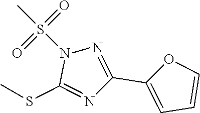 [SMILES: CSc1nc(-c2ccco2)nn1S(C)(=O)=O]

or a salt thereof.

In some embodiments, a weight % of the compound in the composition ranges from about 0.0001% to about 10% by weight relative to a total weight of the composition. In some embodiments, a weight % of the compound in the composition is at least about 0.0001, 0.0005, 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10% by weight relative to a total weight of the composition. In some embodiments, a weight % of the compound in the composition is at least about 0.0001% by weight relative to a total weight of the composition. In some embodiments, a weight % of the compound in the composition is at least about 0.0005% by weight relative to a total weight of the composition. In some embodiments, a weight % of the compound in the composition is at least about 0.001% by weight relative to a total weight of the composition. In some embodiments, a weight % of the compound in the composition is at least about 0.005% by weight relative to a total weight of the composition. In some embodiments, a weight % of the compound in the composition is at least about 0.01% by weight relative to a total weight of the composition. In some embodiments, a weight % of the compound in the composition is at least about 0.02% by weight relative to a total weight of the composition. In some embodiments, a weight % of the compound in the composition is at least about 0.03% by weight relative to a total weight of the composition. In some embodiments, a weight % of the compound in the composition is at least about 0.04% by weight relative to a total weight of the composition. In some embodiments, a weight % of the compound in the composition is at least about 0.05% by weight relative to a total weight of the composition. In some embodiments, a weight % of the compound in the composition is at least about 0.06% by weight relative to a total weight of the composition. In some embodiments, a weight % of the compound in the composition is at least about 0.07% by weight relative to a total weight of the composition. In some embodiments, a weight % of the compound in the composition is at least about 0.08% by weight relative to a total weight of the composition. In some embodiments, a weight % of the compound in the composition is at least about 0.09% by weight relative to a total weight of the composition. In some embodiments, a weight % of the compound in the composition is at least about 0.1% by weight relative to a total weight of the composition. In some embodiments, a weight % of the compound in the composition is at least about 0.2% by weight relative to a total weight of the composition. In some embodiments, a weight % of the compound in the composition is at least about 0.3% by weight relative to a total weight of the composition. In some embodiments, a weight % of the compound in the composition is at least about 0.4% by weight relative to a total weight of the composition. In some embodiments, a weight % of the compound in the composition is at least about 0.5% by weight relative to a total weight of the composition. In some embodiments, a weight % of the compound in the composition is at least about 0.6% by weight relative to a total weight of the composition. In some embodiments, a weight % of the compound in the composition is at least about 0.7% by weight relative to a total weight of the composition. In some embodiments, a weight % of the compound in the composition is at least about 0.8% by weight relative to a total weight of the composition. In some embodiments, a weight % of the compound in the composition is at least about 0.9% by weight relative to a total weight of the composition. In some embodiments, a weight % of the compound in the composition is at least about 1% by weight relative to a total weight of the composition. In some embodiments, a weight % of the compound in the composition is at most about 0.0001, 0.0005, 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10% by weight relative to a total weight of the composition. In some embodiments, a weight % of the compound in the composition is at most about 0.0001% by weight relative to a total weight of the composition. In some embodiments, a weight % of the compound in the composition is at most about 0.0005% by weight relative to a total weight of the composition. In some embodiments, a weight % of the compound in the composition is at most about 0.001% by weight relative to a total weight of the composition. In some embodiments, a weight % of the compound in the composition is at most about 0.005% by weight relative to a total weight of the composition. In some embodiments, a weight % of the compound in the composition is at most about 0.01% by weight relative to a total weight of the composition. In some embodiments, a weight % of the compound in the composition is at most about 0.02% by weight relative to a total weight of the composition. In some embodiments, a weight % of the compound in the composition is at most about 0.03% by weight relative to a total weight of the composition. In some embodiments, a weight % of the compound in the composition is at most about 0.04% by weight relative to a total weight of the composition. In some embodiments, a weight % of the compound in the composition is at most about 0.05% by weight relative to a total weight of the composition. In some embodiments, a weight % of the compound in the composition is at most about 0.06% by weight relative to a total weight of the composition. In some embodiments, a weight % of the compound in the composition is at most about 0.07% by weight relative to a total weight of the composition. In some embodiments, a weight % of the compound in the composition is at most about 0.08% by weight relative to a total weight of the composition. In some embodiments, a weight % of the compound in the composition is at most about 0.09% by weight relative to a total weight of the composition. In some embodiments, a weight % of the compound in the composition is at most about 0.1% by weight relative to a total weight of the composition. In some embodiments, a weight % of the compound in the composition is at most about 0.2% by weight relative to a total weight of the composition. In some embodiments, a weight % of the compound in the composition is at most about 0.3% by weight relative to a total weight of the composition. In some embodiments, a weight % of the compound in the composition is at most about 0.4% by weight relative to a total weight of the composition. In some embodiments, a weight % of the compound in the composition is at most about 0.5% by weight relative to a total weight of the composition. In some embodiments, a weight % of the compound in the composition is at most about 0.6% by weight relative to a total weight of the composition. In some embodiments, a weight % of the compound in the composition is at most about 0.7% by weight relative to a total weight of the composition. In some embodiments, a weight % of the compound in the composition is at most about 0.8% by weight relative to a total weight of the composition. In some embodiments, a weight % of the compound in the composition is at most about 0.9% by weight relative to a total weight of the composition. In some embodiments, a weight % of the compound in the composition is at most about 1% by weight relative to a total weight of the composition. In some embodiments, a weight % of the compound in the composition ranges from about 0.0001 to 10, 0.0005 to 9, 0.001 to 8, 0.005 to 7, 0.01 to 6, 0.02 to 5, 0.03 to 4, 0.04 to 3, 0.05 to 2, 0.06 to 1, 0.07 to 0.9, 0.08 to 0.8, 0.09 to 0.7, 0.1 to 0.6, 0.2 to 0.5, or 0.3 to 0.4% by weight relative to a total weight of the composition. In some embodiments, the weight % of the compound in the composition ranges from about 0.0001% to about 1.0% by weight relative to a total weight of the composition. In some embodiments, the weight % of the compound in the composition ranges from about 0.001% to about 2.0% by weight relative to a total weight of the composition.

In some embodiments, the composition is a pharmaceutical composition. In some embodiments, the pharmaceutical composition is formulated into a variety of topically administrable compositions, including but are not limited to a solution, a suspension, an ointment, a paste, a lotion, a cream, a gel, a balm, or a medicated stick. In some embodiments, the composition further comprises at least one additive selected from the group consisting of pharmaceutically acceptable carriers, excipients, adjuvants, and diluents. In some embodiments, the excipients include but are not limited to a solvent, an antibacterial agent, a buffer, or an isotonic agent. In some embodiments, the buffer is selected from phosphate-buffered saline (PBS), acetate buffer, citrate buffer, tris buffer, glutamate buffer, phosphate buffer, sodium bicarbonate, and ammonium chloride. In some embodiments, the composition comprises an acid to maintain buffer conditions. In some embodiments, the composition comprises citric acid to maintain buffer conditions. In some embodiments, the composition comprises an acid to maintain a pH condition. In some embodiments, the composition comprises citric acid to maintain a pH condition. In some embodiments, pH of the composition ranges from about 2 to about 6. In some embodiments, pH of the composition ranges from about 4 to about 5. In some embodiments, pH of the composition ranges from about 3 to about 5. In some embodiments, pH of the composition ranges from about 2 to about 4. In some embodiments, pH of the composition is not lower than 2, 3, 4, 5, or 6. In some embodiments, pH of the composition is not higher than 8, 7, 6, 5, 4, or 3. In some embodiments, the composition comprises compounds that have poly-ethoxylated groups including polyethylene glycol (PEG)-8 Dimethicone, PEG-40 Hydrogenated Castor Oil, PEG-8 and other PEG compounds.

In some embodiments, the composition is a cosmetic composition. In some embodiments, the cosmetic composition is formulated as a various forms, including but not limited to a toner, an emulsion, a cream, a gel, a shampoo, a soap, a serum, a spray, an oil, an ointment, a paste, a lotion, a balm, or a suspension. In some embodiments, the composition further comprises at least one additive selected from the group consisting of cosmetically acceptable carriers, excipients, adjuvants, and diluents. In some embodiments, the excipients include but are not limited to a solvent, an antibacterial agent, a buffer, or an isotonic agent. In some embodiments, the composition comprises an acid to maintain buffer conditions. In some embodiments, the composition comprises citric acid to maintain buffer conditions. In some embodiments, the composition comprises an acid to maintain a pH condition. In some embodiments, the composition comprises citric acid to maintain a pH condition. In some embodiments, pH of the composition ranges from about 2 to about 6. In some embodiments, pH of the composition ranges from about 4 to about 5. In some embodiments, pH of the composition ranges from about 3 to about 5. In some embodiments, pH of the composition ranges from about 2 to about 4. In some embodiments, pH of the composition not lower than 2, 3, 4, 5, or 6. In some embodiments, pH of the composition is not higher than 8, 7, 6, 5, 4, or 3. In some embodiments, the composition comprises compounds that have poly-ethoxylated groups including polyethylene glycol (PEG)-8 Dimethicone, PEG-40 Hydrogenated Castor Oil, PEG-8 and other PEG compounds.

In some embodiments, the composition comprises one or more compounds or salts thereof disclosed herein. In some embodiments, the composition comprises one compound or salt thereof disclosed herein. In some embodiments, the composition comprises two compounds or salts thereof disclosed herein. In some embodiments, the composition comprises three compounds or salts thereof disclosed herein.

In some embodiments, the composition is used for treating hair loss or hair thinning of a subject in need thereof. In some embodiments, the hair loss is selected from androgenic alopecia, alopecia areata, androgenetic alopecia, gynecologic alopecia, postpartum alopecia, seborrheic alopecia, non-rigid alopecia, senile alopecia, chemotherapy-induced alopecia, radiation-induced alopecia, male-pattern baldness, female-pattern baldness, cicatricial alopecia, alopecia areata telogen effluvium, traction alopecia, anagen effluvium, and combinations thereof. In some embodiments, the composition promotes hair strength, hair growth, hair restoration, hair thickening, or combinations thereof of the subject. In some embodiments, the composition increases hair density, hair growth rate, or combination thereof of the subject. In some embodiments, the hair includes, but not limited to a scalp hair, an eyelash hair, an eyebrow hair, a facial hair, or a combination thereof. The composition may include any aspect or combination of aspects from any of the compounds disclosed herein.

TABLE 2 to TABLE 11 provide examples of the composition described herein. The terms of A, B, C, D, and E in TABLE 2 to TABLE 11 represent example ranges of percent weights of each ingredient. For instance, A refers to values less than about 0.05%, B refers to values equal to or larger than about 0.05% and less than about 1%, C represents values equal to or larger than about 1% and less than about 10%, D refers to values equal to or larger than about 10% and less than about 25%, and E represents values equal to or larger than about 25%. Based on these examples, a composition may include any aspect in TABLE 2 to TABLE 11 in an amount shown therein.

In some embodiments, the composition disclosed herein maintains chemical stability of the compound of Formula (I) during storage at room temperature for at least about 1, 2, 3, 4, 5, 6, 7, 8, 9, 10, 11, 12, 16, 20, 24, 36, 48, or 60 months. In some embodiments, the composition disclosed herein maintains chemical stability of the compound of Formula (I) during storage at room temperature for at most about 1, 2, 3, 4, 5, 6, 7, 8, 9, 10, 11, 12, 16, 20, 24, 36, 48, or 60 months. In some embodiments, the composition disclosed herein maintains chemical stability of the compound of Formula (I) during storage at room temperature for about from 1 month to 60 months, from 2 months to 48 months, from 3 months to 36 months, from 4 months to 24 months, from 5 months to 20 months, from 6 months to 16 months, from 7 months to 12 months, from 8 months to 11 months, from 9 months to 10 months. In some embodiments, the composition disclosed herein maintains chemical stability of the compound I-1 during storage at room temperature for at least about 1, 2, 3, 4, 5, 6, 7, 8, 9, 10, 11, 12, 16, 20, 24, 36, 48, or 60 months. In some embodiments, the composition disclosed herein maintains chemical stability of the compound I-1 during storage at room temperature for at most about 1, 2, 3, 4, 5, 6, 7, 8, 9, 10, 11, 12, 16, 20, 24, 36, 48, or 60 months. In some embodiments, the composition disclosed herein maintains chemical stability of the compound I-1 during storage at room temperature for about from 1 month to 60 months, from 2 months to 48 months, from 3 months to 36 months, from 4 months to 24 months, from 5 months to 20 months, from 6 months to 16 months, from 7 months to 12 months, from 8 months to 11 months, from 9 months to 10 months.

In some embodiments, the composition disclosed herein preserves the chemical structure of the compound of Formula (I). In some embodiments, the composition disclosed herein prevents hydrolysis of the compound of Formula (I). In some embodiments, the composition disclosed herein prevents degradation of the compound of Formula (I). In some embodiments, the composition disclosed herein preserves the chemical structure of the compound I-1. In some embodiments, the composition disclosed herein prevents hydrolysis of the compound I-1. In some embodiments, the composition disclosed herein prevents degradation of the compound I-1.

TABLE 2
Example of composition

| Ingredient | % Weight |
|---|---|
| Aqua/Eau/Water | E |
| Ethanol | D |
| Propylene Glycol | D |
| Glycerin | C |
| Propanediol | C |
| Caffeine | B |
| Serenoa Serrulata Extract | B |
| Pyrus Malus Extract | B |
| Phenoxyethanol | B |
| l-Arginine | B |
| l-Lysine | B |
| Inositol | B |
| Aloe Barbadensis Leaf Juice | A |
| Keratin Amino Acid | A |
| A compound of Formula (I) | A |
| Ethylhexylglycerin | A |
| Pantothenic Acid | A |
| l-Methionine | A |
| Xanthan Gum | A |
| Niacin | A |
| n-Acetylcysteine | A |
| Biotin | A |
| Thiamine HCI (Vitamin B1) | A |

TABLE 3
Example of composition

| Ingredient | % Weight |
|---|---|
| Water/Aqua/Eau | E |
| Propanediol | D |
| Alcohol Denat. | D |
| Glycerin | C |
| Dimethyl Isosorbide | C |
| Ethoxydiglycol | C |
| Polysorbate 20 | C |
| PEG-40 Castor Oil | C |
| Panthenol | B |
| Phenoxyethanol | B |
| Malus Domestica Fruit Cell Culture Extract | B |
| (and) Xanthan Gum (and) Glycerin (and) | |
| Lecithin (and) Phenoxyethanol (and) Water | |
| (Aqua) | |
| Isopentyldiol (and) Trifolium Pratense (Red | B |
| Clover) Flower Extract | |
| Thiamine HCl | B |
| Ethylhexylglycerin | B |
| Allantoin | B |
| Caffeine | B |
| Inositol | B |
| Dipotassium Glycyrrhizate | B |
| Tetrahydrodiferuloylmethane, | B |
| Tetrahydrodemethoxydiferuloylmethane, | |
| Tetrahydrobisdemethoxydiferuloylmethane | |
| Serenoa Serrulata Fruit Extract | B |
| Panax Ginseng Root Extract | B |
| Resveratrol | B |
| Aloe Barbadensis Leaf Juice | B |
| Sodium Phytate | B |
| Niacinamide | B |
| Raspberry Ketone | B |
| Ganoderma Lucidum Extract | B |
| A compound of Formula (I) | A |
| LarI Gmelinii (Larch) Wood Extract | A |
| Pyrus Malus (Apple) Fruit Extract | A |
| Camellia Sinensis Leaf Extract | A |
| Piper Nigrum Fruit Extract | A |
| Pinus Pinaster Bark Extract | A |
| Citrus Paradisi (Grapefruit) Peel Extract | A |
| Olea Europaea (Olive) Leaf Extract | A |

TABLE 4
Example of composition

| Ingredient | % Weight |
|---|---|
| Water/Aqua/Eau | E |
| Propanediol | D |
| Alcohol Denat. | D |
| Dimethyl Isosorbide | D |
| Glycerin | C |
| Ethoxydiglycol | C |
| PEG-40 Castor Oil | C |
| Panthenol | B |
| Phenoxyethanol | B |
| Polysorbate 20 | B |
| Malus Domestica Fruit Cell Culture Extract | B |
| (and) Xanthan Gum (and) Glycerin (and) | |
| Lecithin (and) Phenoxyethanol (and) Water | |
| (Aqua) | |
| Isopentyldiol (and) Trifolium Pratense (Red | B |
| Clover) Flower Extract | |
| Ethylhexylglycerin | B |
| Allantoin | B |
| Inositol | B |
| Dipotassium Glycyrrhizate | B |
| Disodium EDTA | B |
| Serenoa Serrulata Fruit Extract | B |
| Resveratrol | B |
| Tetrahydrodiferuloylmethane, | B |
| Tetrahydrodemethoxydiferuloylmethane, | |
| Tetrahydrobisdemethoxydiferuloylmethane | |
| Aloe Barbadensis Leaf Juice | B |
| Panax Ginseng Root Extract | A |
| Citric Acid | A |
| A compound of Formula (I) | A |
| Ganoderma Lucidum Extract | A |
| Piper Nigrum Fruit Extract | A |
| Pinus Pinaster Bark Extract | A |
| Olea Europaea (Olive) Leaf Extract | A |

TABLE 5
Example of composition

| Ingredient | % Weight |
|---|---|
| Water/Aqua/Eau | E |
| Alcohol Denat. | D |
| Dimethyl Isosorbide | D |
| Glycerin | C |
| Propanediol | C |
| Polysorbate 20 | C |
| Ethoxydiglycol | C |
| PEG-40 Hydrogenated Castor Oil | C |
| d-Panthenol | B |
| Phenoxyethanol | B |
| Hydroxyethylcellulose | B |
| Malus Domestica Fruit Cell Culture Extract | B |
| (and) Xanthan Gum (and) Glycerin (and) | |
| Lecithin (and) Phenoxyethanol (and) Water | |
| (Aqua) | |
| Ethylhexylglycerin | B |
| Allantoin | B |
| Dipotassium Glycyrrhizate | B |
| Pyrus Malus Skin Extract | B |
| Aloe Barbadensis Leaf (Aloe Vera) Extract | B |
| Caffeine | B |
| Inositol | B |
| Resveratrol | B |
| Disodium EDTA | B |
| A compound of Formula (I) | A |
| Citric Acid | A |
| Citrus Paradisi (Grapefruit) Peel Extract | A |
| Olea Europaea (Olive) Leaf Extract | A |

TABLE 6
Example of composition

| Ingredient | % Weight |
|---|---|
| Water/Aqua/Eau | E |
| Propanediol | D |
| Butylene Glycol | D |
| Dimethyl Isosorbide | C |
| Polysorbate 20 | C |
| Ethoxydiglycol | C |
| Glycerin | C |
| PEG-40 Castor Oil | C |
| Pisum Sativum (Pea) Sprout Extract (and) | C |
| Phenoxyethanol (and) Sodium Benzoate | |
| (and) Water (Aqua) | |
| Malus Domestica Fruit Cell Culture Extract | C |
| (and) Xanthan Gum (and) Glycerin (and) | |
| Lecithin (and) Phenoxyethanol (and) Water | |
| (Aqua) | |
| PEG-8 Dimethicone | C |
| Phenoxyethanol | B |
| Hydroxyethylcellulose | B |
| Panthenol | B |
| Glycerin (and) Water (and) Calendula | B |
| Officinalis Extract | |
| Disodium EDTA | B |
| Dipotassium Glycyrrhizate | B |
| Ethylhexylglycerin | B |
| Citric Acid | B |
| Tetrahydrodiferuloylmethane, | B |
| Tetrahydrodemethoxydiferuloylmethane, | |
| Tetrahydrobisdemethoxydiferuloylmethane | |
| Panax Ginseng Root Extract | A |
| A compound of Formula (I) | A |

TABLE 7
Example of composition

| Ingredient | % Weight |
|---|---|
| Water/Aqua/Eau | E |
| Alcohol Denat. | D |
| Butylene Glycol | C |
| Propanediol | C |
| Fructose (and) Glycerin (and) Water (and) | C |
| Sechium Edule Fruit Extract | |
| Glycerin (and) Water (and) Hippophae | C |
| Rhamnoides Fruit Extract | |
| Pisum Sativum (Pea) Sprout Extract (and) | C |
| Phenoxyethanol (and) Sodium Benzoate | |
| (and) Water (Aqua) | |
| Water (and) Furcellaria Lumbricalis Extract | C |
| Phenoxyethanol | B |
| Panthenol | B |
| Glycerin (and) Water (and) Calendula | B |
| Officinalis Extract | |
| Isopentyldiol (and) Trifolium Pratense (Red | B |
| Clover) Flower Extract | |
| Dipotassium Glycyrrhizate | B |
| Resveratrol | B |
| Ethylhexylglycerin | B |
| Aloe Barbadensis Leaf Juice | B |
| Citric Acid | B |
| Panax Ginseng Root Extract | A |
| A compound of Formula (I) | A |

TABLE 8
Example of composition

| Ingredient | % Weight |
|---|---|
| Caprylic/Capric Triglycerides | E |
| Coco-Caprylate | D |
| Isodecyl Neopentanoate | D |
| Hydrogenated Vegetable Oil | C |
| Punica Granatum (Pomegranate) Seed Oil | C |
| Dimethyl Isosorbide | C |
| Squalane | C |
| Avocado Oil | C |
| Argania Spinosa (Argan) Kernel Oil | C |
| Apricot Kernel Oil | C |
| Vitis Vinifera (Grapeseed) Seed Oil | C |
| Ethoxydiglycol | C |
| PEG-40 Hydrogenated Castor Oil | B |
| Sorbitan Laurate | B |
| Tocopherol | B |
| A compound of Formula (I) | B |

TABLE 9
Example of composition

| Ingredient | % Weight |
|---|---|
| Argania Spinosa (Argan) Kernel Oil | E |
| Dimethyl Isosorbide | D |
| Octyldodecanol | C |
| Ricinus Communis (Castor) Seed Oil | C |
| Ethoxydiglycol | C |
| PEG-40 Hydrogenated Castor Oil | B |
| Polysorbate 20 | B |
| Tocopheryl Acetate | B |
| A compound of Formula (I) | B |
| Citrus Paradisi (Grapefruit) Peel Extract | A |
| Olea Europaea (Olive) Leaf Extract | A |

TABLE 10
Example of composition

| Ingredient | % Weight |
|---|---|
| Caprylic/Capric Triglycerides | E |
| Coco-Caprylate | D |
| Dimethyl Isosorbide | D |
| Punica Granatum (Pomegranate) Seed Oil | C |
| Cucurbita Pepo (Pumpkin) Seed Oil | C |
| Argania Spinosa (Argan) Kernel Oil | C |
| Borago Officinalis (Borage) Seed Oil | C |
| Ethoxydiglycol | C |
| Octyldodecanol | C |
| PEG-40 Hydrogenated Castor Oil | B |
| Sorbitan Laurate | B |
| Ricinus Communis (Castor) Seed Oil | B |
| Vitis Vinifera (Grapeseed) Seed Oil | B |
| Tocopherol | B |
| Brassica Oleracea Italica (Broccoli) Seed | B |
| Oil | |
| A compound of Formula (I) | B |
| Pinus Pinaster Bark Extract | A |
| Olea Europaea (Olive) Leaf Extract | A |

TABLE 11
Example of composition

| Ingredient | % Weight |
|---|---|
| Water/Aqua/Eau | E |
| Denatured Ethanol | D |
| 1,3-Propanediol | D |
| Dimethyl isosorbide | C |
| Ethoxydiglycol [i.e. 2-(2-Ethoxyethoxy)ethanol] | C |
| Glycerin | C |
| PEG-40 Castor Oil | B |
| Phenoxyethanol | B |
| Ethylhexylglycerin | B |
| Disodium EDTA | B |
| Citric acid | B |
| A compound of Formula (I) | B |
| Furanyl Methylthio Methylsulfanyltriazole (1H- | A |
| 1,2,4-Triazole,3-(2-furanyl)-1-(methylsulfonyl)-5- | |
| (methylthio)-) | |

### METHODS

Also provided herein include methods for treating hair loss or hair thinning of a subject in need thereof with the compound(s) or salt(s) disclosed herein. In certain aspects, disclosed herein is a method for preventing or treating hair loss or hair thinning. In some embodiments, the method is for treating hair loss. In some embodiments, the method is for treating hair thinning. In some embodiments, the method comprises administering to a subject in need thereof a composition comprising a compound having a structure of Formula (I) or salt thereof described herein, or a formulation described herein. The compound(s) or salt(s) thereof may have a structural Formula of (I);

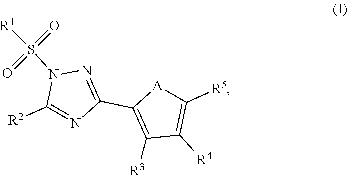

or a salt thereof,
wherein:
R¹ is selected from hydrogen, —OH, C₁₋₆ alkyl, and C₁₋₆ haloalkyl;
R² is selected from hydrogen, —OH, C₁₋₆ alkoxy, C₁₋₆ haloalkoxy, —SH, —S-alkyl, —S-haloalkyl, and —S—(CH₂CH₂—O)ₙ—R^{a}; wherein
n is an integer from 0 to 6, and
R^{a} is C₁₋₆ alkyl or C₁₋₆ haloalkyl;
A is NH, O or S; and
R³, R⁴, and R⁵ are independently selected from hydrogen, —OH, C₁₋₆ alkyl, and C₁₋₆ haloalkyl.

In some embodiments, R¹ is C₁₋₆ alkyl. In some embodiments, R¹ is selected from —CH₃, —(CH₂)₂CH₃, and —(CH₂)₄CH₃. In some embodiments, R¹ is —CH₃. In some embodiments, R¹ is —(CH₂)₂CH₃. In some embodiments, R¹ is —(CH₂)₄CH₃. In some embodiments, R² is —S-alkyl. In some embodiments, R² is —S—CH₃. In some embodiments, R² is —S—CH₂CH₃. In some embodiments, R² is —S—(CH₂CH₂—O)ₙ—R^{a}. In some embodiments, n is 1 or 2. In some embodiments, R^{a} is C₁₋₆ alkyl. In some embodiments, R^{a} is —CH₃. In some embodiments, R² is —S—CH₂CH₂—O—CH₃. In some embodiments, R² is —S—(CH₂CH₂-O)₂—CH₃. In some embodiments, A is O. In some embodiments, R³ is hydrogen. In some embodiments, R⁴ is hydrogen. In some embodiments, R⁵ is hydrogen.

In some embodiments, the compound of Formula (I) is selected from:

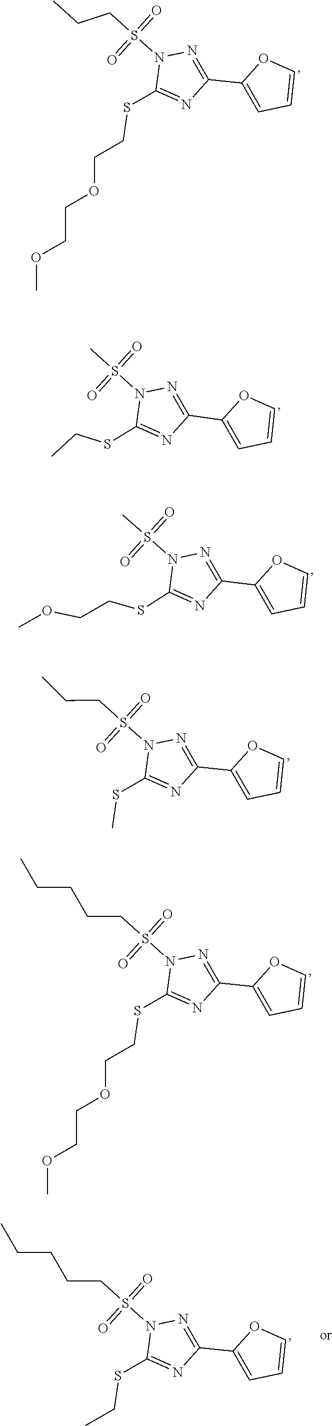 [SMILES: CCCS(=O)(=O)n1nc(-c2ccco2)nc1SCCOCCOC ; CCSc1nc(-c2ccco2)nn1S(C)(=O)=O ; COCCSc1nc(-c2ccco2)nn1S(C)(=O)=O ; CCCS(=O)(=O)n1nc(-c2ccco2)nc1SC ; CCCCCS(=O)(=O)n1nc(-c2ccco2)nc1SCCOCCOC ; CCCCCS(=O)(=O)n1nc(-c2ccco2)nc1SCC]

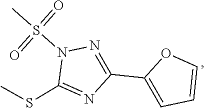 [SMILES: CSc1nc(-c2ccco2)nn1S(C)(=O)=O]

or a salt thereof.

In some embodiments, the compound of Formula (I) is

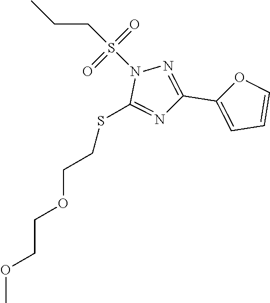 [SMILES: CCCS(=O)(=O)n1nc(-c2ccco2)nc1SCCOCCOC]

or a salt thereof. In some embodiments, the compound of Formula (I) is

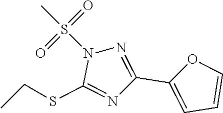 [SMILES: CCSc1nc(-c2ccco2)nn1S(C)(=O)=O]

or a salt thereof. In some embodiments, the compound of Formula (I) is

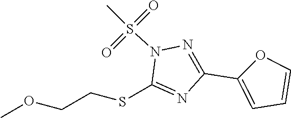 [SMILES: COCCSc1nc(-c2ccco2)nn1S(C)(=O)=O]

or a salt thereof. In some embodiments, the compound of Formula (I) is

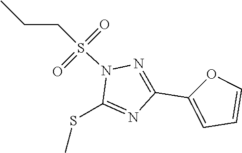 [SMILES: CCCS(=O)(=O)n1nc(-c2ccco2)nc1SC]

or a salt thereof. In some embodiments, the compound of Formula (I) is

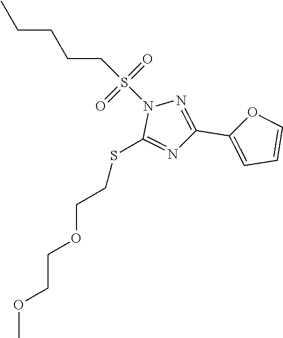 [SMILES: CCCCCS(=O)(=O)n1nc(-c2ccco2)nc1SCCOCCOC]

or a slat thereof. In some embodiments, the compound of Formula (I) is

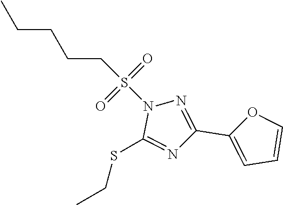 [SMILES: CCCCCS(=O)(=O)n1nc(-c2ccco2)nc1SCC]

or a salt thereof. In some embodiments, the compound of Formula (I) is

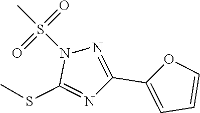 [SMILES: CSc1nc(-c2ccco2)nn1S(C)(=O)=O]

or a salt thereof.

In some embodiments, a weight % of the compound in the composition ranges from about 0.0001% to about 10% by weight relative to the total weight of the composition. In some embodiments, a weight % of the compound in the composition is at least about 0.0001, 0.0005, 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10% by weight relative to the total weight of the composition. In some embodiments, a weight % of the compound in the composition is at most about 0.0001, 0.0005, 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10% by weight relative to the total weight of the composition. In some embodiments, a weight % of the compound in the composition ranges from about 0.0001 to 10, 0.0005 to 9, 0.001 to 8, 0.005 to 7, 0.01 to 6, 0.02 to 5, 0.03 to 4, 0.04 to 3, 0.05 to 2, 0.06 to 1, 0.07 to 0.9, 0.08 to 0.8, 0.09 to 0.7, 0.1 to 0.6, 0.2 to 0.5, or 0.3 to 0.4% by weight relative to the total weight of the composition. In some embodiments, a weight % of the compound in the composition ranges from about 0.01% to about 2.0%.

In some embodiments, the method comprises administering to a subject in need thereof a composition comprising one or more compounds or salts thereof disclosed herein. In some embodiments, administering the composition comprising one or more compounds or salts thereof, as disclosed herein, may enhance the treatment effect for hair loss or hair thinning in the subject. In some embodiments, the composition may comprise two compounds or salts thereof disclosed herein. In some embodiments, the composition may comprise three compounds or salts thereof disclosed herein.

A diagnosis of hair loss or hair thinning may or may not have been made. In some embodiments of any method described herein, the subject may have been diagnosed with hair loss or hair thinning. In some embodiments of any method described herein, the subject may not have been diagnosed with hair loss or hair thinning.

In some embodiments, the composition is a pharmaceutical composition. In some embodiments, the pharmaceutical composition further comprises at least one additive selected from the group consisting of pharmaceutically acceptable carriers, excipients, adjuvants, and diluents. In some embodiments, the pharmaceutical composition is formulated into a variety of topically administrable compositions, including but are not limited to a solution, a suspension, an ointment, a paste, a lotion, a cream, a gel, a balm, or a medicated stick.

In some embodiments, the pharmaceutical composition is administered one to three times a day. In some embodiments, the pharmaceutical composition is administered once a day. In some embodiments, the pharmaceutical composition is administered two times a day. In some embodiments, the pharmaceutical composition is administered three times a day. In some embodiments, the pharmaceutical composition is administered every other day. In some embodiments, the pharmaceutical composition is administered for at least two consecutive days. In some embodiments, the pharmaceutical composition is administered for at least three consecutive days. In some embodiments, the pharmaceutical composition is administered for at least four consecutive days. In some embodiments, the pharmaceutical composition is administered for at least five consecutive days. In some embodiments, the pharmaceutical composition is administered for at least seven consecutive days.

In some embodiments, the pharmaceutical composition is administered at least for about 2 days, 3 days, 5 days, 7 days, 10 days, 15 days, 20 days, 30 days, 50 days, 60 days, 80 days, 90 days, 5 months, 6 months, 7 months, 8 months, 9 months, 10 months, 11 months, a year, 2 years, or 3 years. In some embodiments, the pharmaceutical composition is administered at most for about 2 days, 3 days, 5 days, 7 days, 10 days, 15 days, 20 days, 30 days, 50 days, 60 days, 80 days, 90 days, 5 months, 6 months, 7 months, 8 months, 9 months, 10 months, 11 months, a year, 2 years, or 3 years. In some embodiments, the pharmaceutical composition is administered from about 2 days to 3 years, 3 days to 2 years, 5 days to a year, 7 days to 11 months, 10 days to 10 months, 15 days to 9 months, 20 days to 8 months, 30 days to 7 months, 50 days to 6 months, 60 days to 5 months, 80 days to 4 months, or 90 days to 3 years.

In some embodiments, the composition is a cosmetic composition. In some embodiments, the cosmetic composition further comprises at least one additive selected from the group consisting of cosmetically acceptable carriers, excipients, adjuvants, and diluents. In some embodiments, the cosmetic composition is formulated as a various forms, including but not limited to a toner, an emulsion, a cream, a gel, a shampoo, a soap, a serum, a spray, or an oil.

In some embodiments, the hair loss is selected from androgenic alopecia, alopecia areata, androgenetic alopecia, gynecologic alopecia, postpartum alopecia, seborrheic alopecia, non-rigid alopecia, senile alopecia, chemotherapy-induced alopecia, radiation-induced alopecia, male-pattern baldness, female-pattern baldness, cicatricial alopecia, alopecia areata telogen effluvium, traction alopecia, anagen effluvium, and combinations thereof. In some embodiments, the method promotes hair strength, hair growth, hair restoration, hair thickening, or combinations thereof of the subject. In some embodiments, the method increases hair density, hair growth rate, or combination thereof of the subject. In some embodiments, the hair includes, but not limited to a scalp hair, an eyelash hair, an eyebrow hair, a facial hair, or a combination thereof.

### KITS

This disclosure also provides a kit comprising the composition disclosed herein. In some embodiments, the kit further comprises an applicator. In some embodiments, the applicator includes but is not limited to a brush, a comb, a cotton swab, a spatula, a spoon, a dropper, a spray nozzle, a squeeze bottle, a tottle, or a combination thereof. In some embodiments, the kit further comprises written instructions for using the composition in a hair treatment.

### EXAMPLES

The disclosure now being generally described, it will be more readily understood by reference to the following examples which are included merely for purposes of illustration of certain aspects and embodiments of the present invention, and are not intended to limit the invention in any way.

### Example 1: Assay of Human Follicle Dermal Papilla Cells (hFDPC) Proliferation

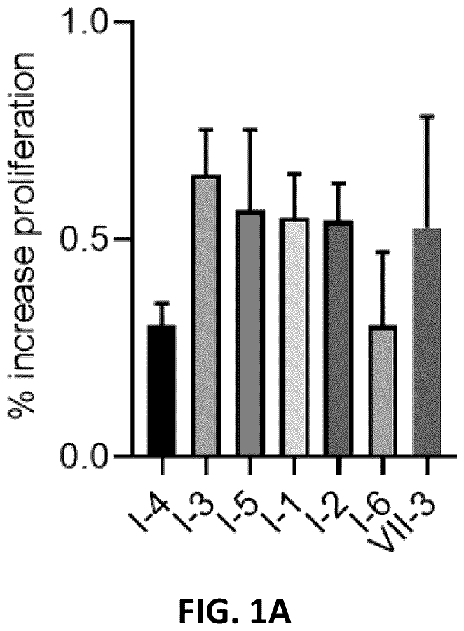

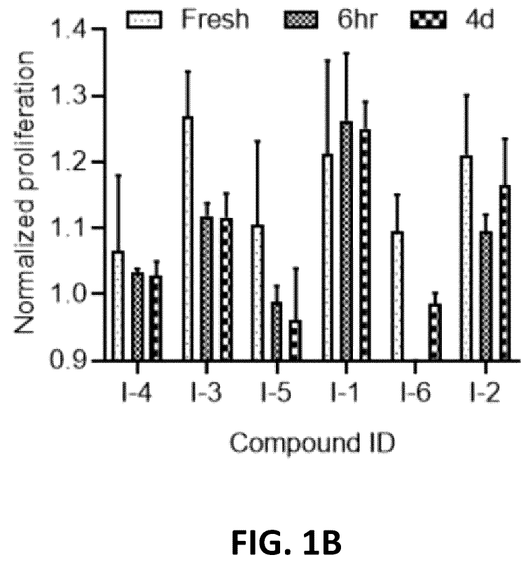

The ability of a compound or salt of the present disclosure to increase proliferation of human hair follicle dermal papilla cells (hFDPC) was tested using a cell viability assay, such as the Promega CellTiter-Glo® Luminescent Cell Viability Assay, MTT cell proliferation assay (ATCC® 30-1010K), or manual cell counting. The compound or salt was added at various concentrations spanning at least a 2-log range. hFDPCs were derived from multiple donors ranging from 25-83 years old and cultured on tissue-treated plastic using predefined growth media supplemented with 6-bromoindirubin-3′-Oxime (BIO), recombinant bone morphogenetic protein-2 (BMP-2), and basic fibroblast growth factor (FGFβ), a combination previously found to preserve in situ dermal papilla gene signatures. TABLE 12 shows the results. In TABLE 12, A represents less than 20% increased proliferation of hFDPCs, B represents a 20-40% increase, and C represents more than a 40% increase in the proliferation of hFDPCs. The results are also shown in FIG. 1A.

TABLE 12
Evaluation of small molecules based on hFDPC proliferation

| | | Increase |
|---|---|---|
| | | Proliferation |
| | | of |
| ID # | Chemical Structure | hFDPCs (%) |
| Control | | 0 |
| (DMSO) | | |
| I-1 | 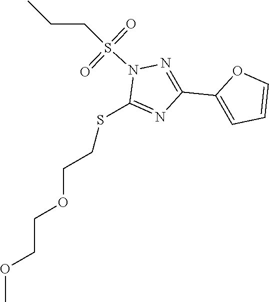 [SMILES: CCCS(=O)(=O)n1nc(-c2ccco2)nc1SCCOCCOC] | C |
| I-2 | 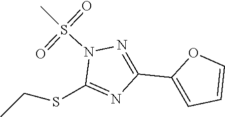 [SMILES: CCSc1nc(-c2ccco2)nn1S(C)(=O)=O] | C |
| I-3 | 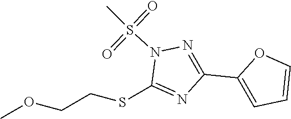 [SMILES: COCCSc1nc(-c2ccco2)nn1S(C)(=O)=O] | C |
| I-4 | 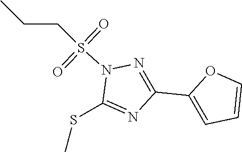 [SMILES: CCCS(=O)(=O)n1nc(-c2ccco2)nc1SC] | B |
| I-5 | 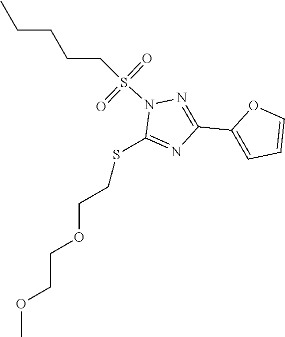 [SMILES: CCCCCS(=O)(=O)n1nc(-c2ccco2)nc1SCCOCCOC] | C |
| I-6 | 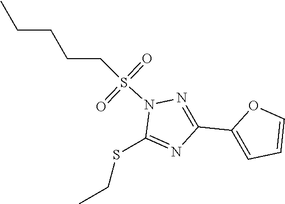 [SMILES: CCCCCS(=O)(=O)n1nc(-c2ccco2)nc1SCC] | B |
| VII-3 | 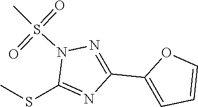 [SMILES: CSc1nc(-c2ccco2)nn1S(C)(=O)=O] | C |

A represents less than 20% of increased proliferation of hFDPCs, B represents 20-40%, and C represents larger than 40% of increased proliferation of hFDPCs.

### Example 2: Assay on Human Keratinocytes

Human adult epidermal keratinocytes were cultured on poly-L-lysine treated plates, and the ability of a compound or salt of the present disclosure to impact proliferation was assessed using a cell viability assay, such as Promega CellTiter-Glo® Luminescent Cell Viability Assay, MTT cell proliferation assay (ATCC® 30-1010K) or cell counting after a 48-hr period. The compound or salt was added at various concentrations spanning at least a single log range.

### Example 3: Assay on Human Dermal Fibroblasts Cells (hDFCs)

The ability of a compound or salt of the present disclosure to impact proliferation of human adult dermal fibroblasts was tested using a cell viability assay, such as Promega CellTiter-Glo® Luminescent Cell Viability Assay, MTT cell proliferation assay (ATCC® 30-1010K) or cell counting after a 48-hr period. The compound or salt was added at various concentrations spanning at least a single log range.

### Example 4: Assay on Human Microvascular Endothelial Cells (hDMECs)

The ability of a compound or salt of the present disclosure to impact proliferation of human adult dermal microvascular endothelial cells was tested using a cell viability assay, such as Promega CellTiter-Glo® Luminescent Cell Viability Assay, MTT cell proliferation assay (ATCC® 30-1010K) or cell counting after a 48-hr period. The compound or salt was added at various concentrations spanning at least a single log range.

### Example 5: Assay on Primary Epidermal Melanocytes; Normal, Human, Adult (HEMa)

The ability of a compound or salt of the present disclosure to impact proliferation of human adult epidermal melanocytes was tested using a cell viability assay, such as Promega

CellTiter-Glo® Luminescent Cell Viability Assay, MTT cell proliferation assay (ATCC® 30-1010K) or cell counting after a 48-hr period. The compound or salt was added at various concentrations spanning at least a single log range. Melanocytes derived from at least two different donors of varying degrees of basal skin pigmentation were used.

### Example 6: Assay on Human Skeletal Muscle Cells

The ability of a compound or salt of the present disclosure to impact proliferation of human skeletal muscle cells was tested using a cell viability assay, such as Promega CellTiter-Glo® Luminescent Cell Viability Assay, MTT cell proliferation assay (ATCC® 30-1010K) or cell counting after a 48-hr period. The compound or salt was added at various concentrations spanning at least a single log range.

### Example 7: Solubility and Stability Studies

The ability of a compound or salt of the present disclosure was found to be fully soluble in several organic cosmetic solvents, including ethanol and dimethyl isosorbide at >10% w/v. Additionally, the ability of a compound or salt of the present disclosure was evaluated at room temperature as well as at 60° C. for varying periods of time (FIG. 1B). The structural stability of compound I-1 was further explored by stirring this compound in 2 different solvent mixtures at elevated temperatures for 2 hours. (TABLE 13). The samples were analyzed by LC-UV after stirring for 2 hrs (FIG. 2). This showed that compound I-1 is very stable in the organic solvents which were used for purification by straight phase column chromatography, with minor degradation in acidic conditions.

TABLE 13
LC-UV Chromatogram Sample Conditions for Compound I-1

| Medium | Conditions | Purity (LC-UV) |
|---|---|---|
| EtOAc/Heptane 1:1 | 80° C., 2 hrs | >99% |
| 0.1% (v/v) HCOOH in H2O/0.1% (v/v) | 80° C., 2 hrs | 95% |
| HCOOH in MeCN 1:1 | | |

### Example 8: In Silico Safety/Toxicology Studies

The in silico safety/toxicology profiles of compounds were evaluated by screening chemical structures against a series of computational models. These models include the Pred-hERG 4.2 cardiotoxicity model and the NeuroDeRisk IL Profiler neurotoxicity model, as well as reproductive and developmental toxicity, carcinogenicity (genotoxic and non-genotoxic), skin sensitization, DNA mutation, and chromosomal aberration models from QSAR Toolbox.

### Example 9: In Vitro Safety/Toxicology Studies

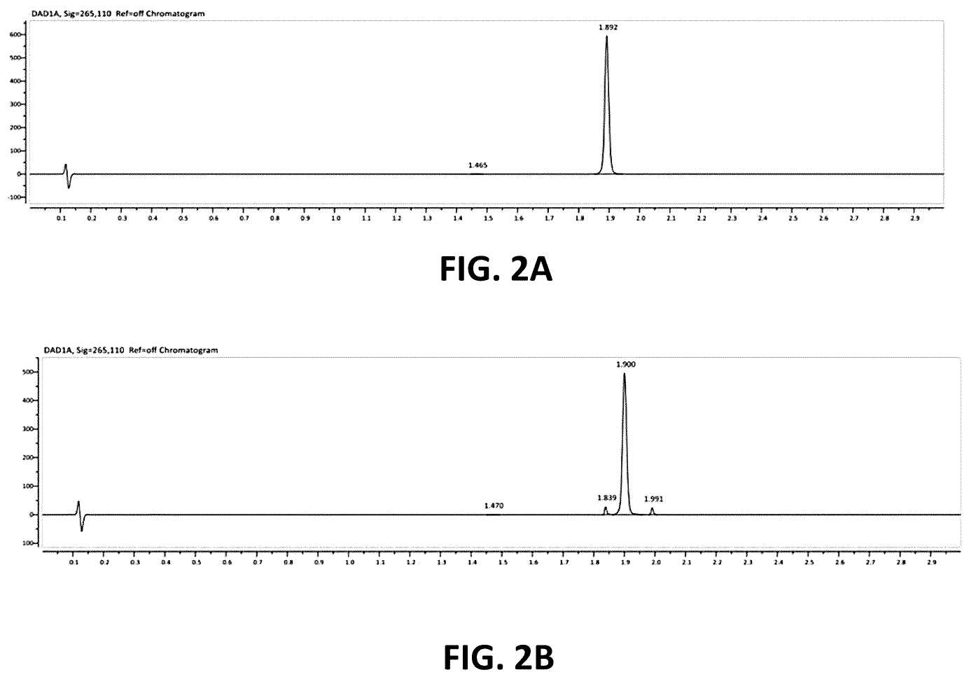

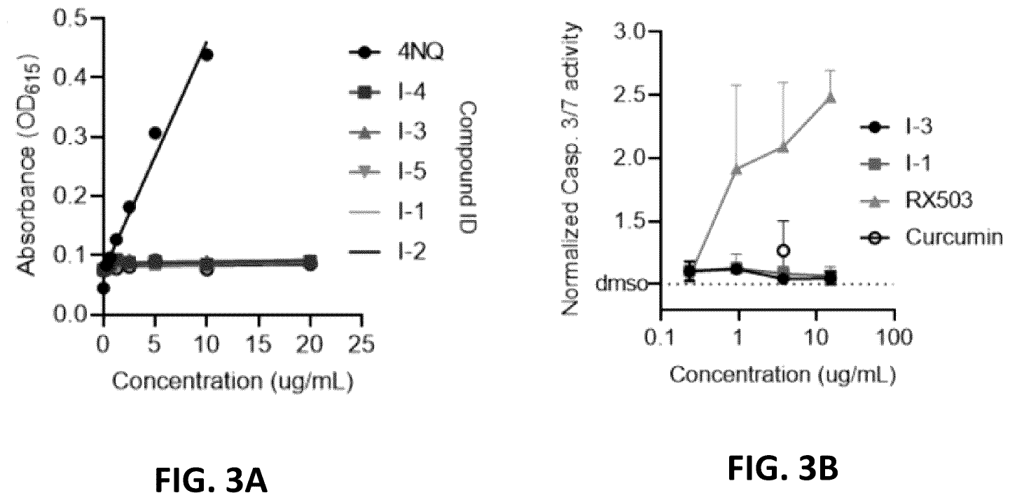

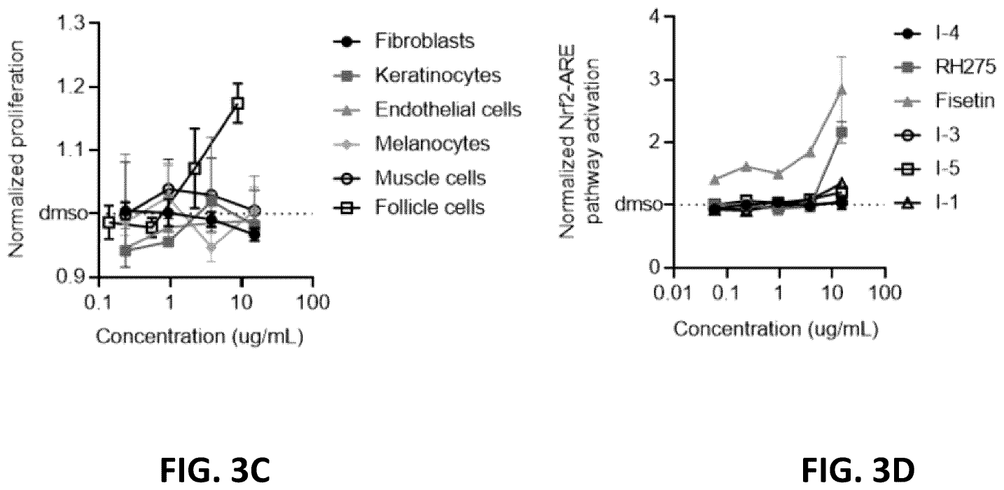

The safety/toxicology profiles of a compound or salt of the present disclosure were evaluated via a series of in vitro genotoxicity assays, including the SOS-chromotest to determine bacterial genotoxicity (FIG. 3A). A standard caspase 3/7 assay was conducted to evaluate general cellular toxicity (FIG. 3B). To further examine the specificity of the compound or salt of the present disclosure, a series of cell viability assays were performed using primary human fibroblasts, keratinocytes, skeletal muscle cells, endothelial cells, and melanocytes (FIG. 3C). A KeratinoSens reporter assay was used to determine any potential skin sensitivity via the activation of a cytoprotective pathway (FIG. 3D).

Additional safety studies including immunological assays using human immature monocyte-derived dendritic cells are to be performed to evaluate any potential immunogenicity of a compound or salt of the present disclosure. Additional safety and toxicity studies include functional cardiotoxicity and neurotoxicity assessments in human iPSC-derived cardiomyocytes and neurons, respectively.

### Example 10: Efficacy Studies

The functional profile of a compound or salt of the present disclosure continues to be investigated in a series of in vitro studies which may include a number of ex vivo models. Transcriptomic and proteomic analyses are to be conducted on relevant genes and proteins with well-established functional roles, including the TGFb and Wnt-signaling pathways, following exposure to a compound or salt of the present disclosure. A clinical efficacy trial is currently being performed with at least 120 human subjects to determine effectiveness of a compound or salt of the present disclosure to promote hair strength, hair growth, hair restoration, hair thickening, or a combination thereof.

### Example 11: Mechanistic Analysis

Using transcriptomics datasets, a series of mechanistic computational analyses are to be performed to identify possible mechanisms of action. These include DE analyses followed by GO term and pathway enrichment to characterize the biological imprint of example compounds. In addition, pseudo-time and cell-cycle analyses are to be performed to probe the developmental effects of these chemicals on target cells.

### Example 12: Biological Assay using Example Compounds

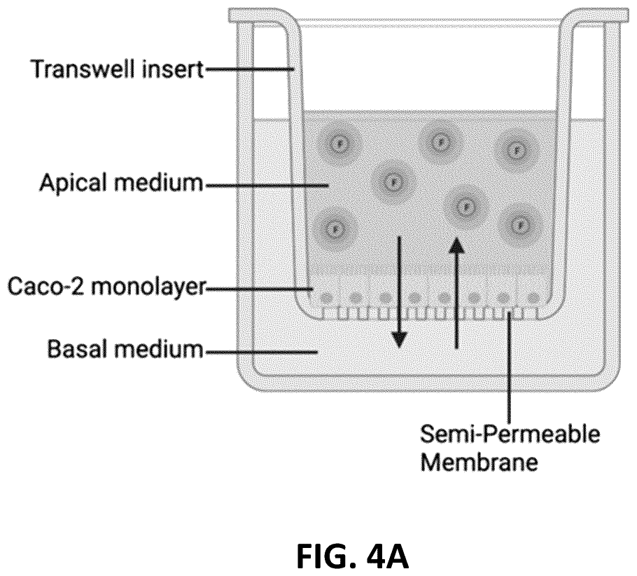

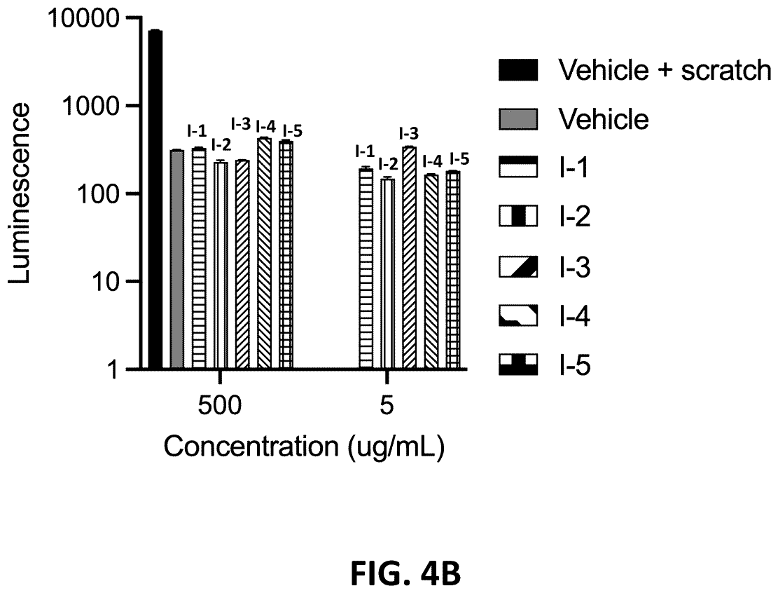

Caco-2 cells were cultured on transwell supports until a monolayer was established, mimicking the intestinal cell barrier. Following this, the cultures were exposed to example compounds (I-1 to I-5) or a vehicle control (0.1% v/v DMSO) on the apical side for a duration of 24 hours, during which they were maintained in a medium supplemented with a fluorescent dye (Lucifer yellow, labeled “F”). FIG. 4A shows a schematic representation of model system.

The apparent permeability of the cell monolayer was evaluated by monitoring raw luminescence signals of a Caco-2 monolayer after dosing with either dimethyl sulfoxide (DMSO) or selected example compounds. A “scratch” refers to a monolayer that has been deliberately disrupted. The results are shown in FIG. 4B. The data represents the mean±standard deviation (s.d.) and is indicative of at least two experimental replicates. No notable permeability was detected in this study. This indicates that the example compounds do not disrupt intestinal cell barrier integrity in a Caco-2 monolayer system.

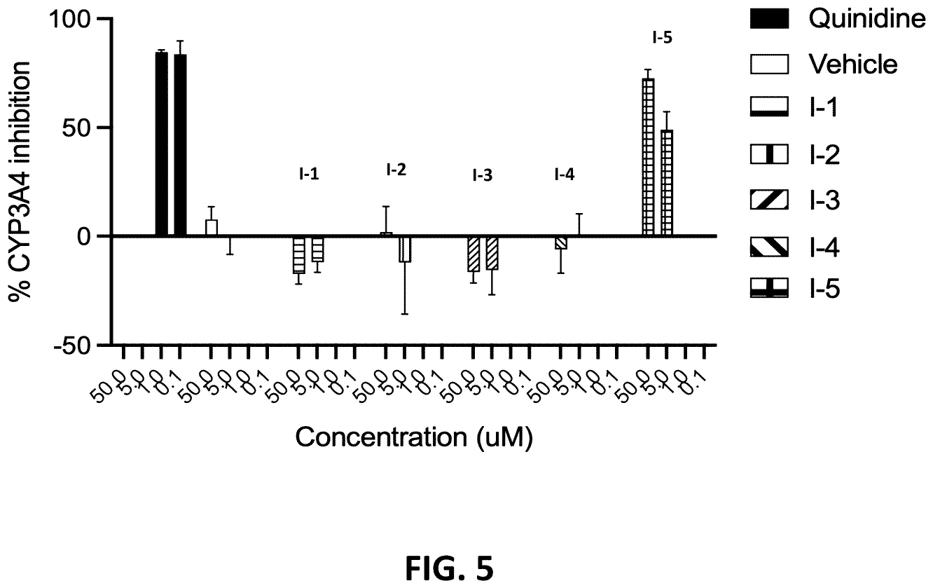

A P450-Glo™ CYP3A4 Assay and Screening System (Promega) was employed to assess the inhibitory potential of the example compounds against CYP3A4 at concentrations of either 50 or 5 μM. As shown in FIG. 5, the majority of the sample compounds demonstrated no significant inhibition of cytochrome P450 3A4 (CYP3A4) activity. Among the tested compounds, only compound I-5 displayed potent inhibitory potential of cytochrome P450 3A4. Vehicle represents 0.1% DMSO and quinidine is a known cytochrome P450 3A4 inhibitor .

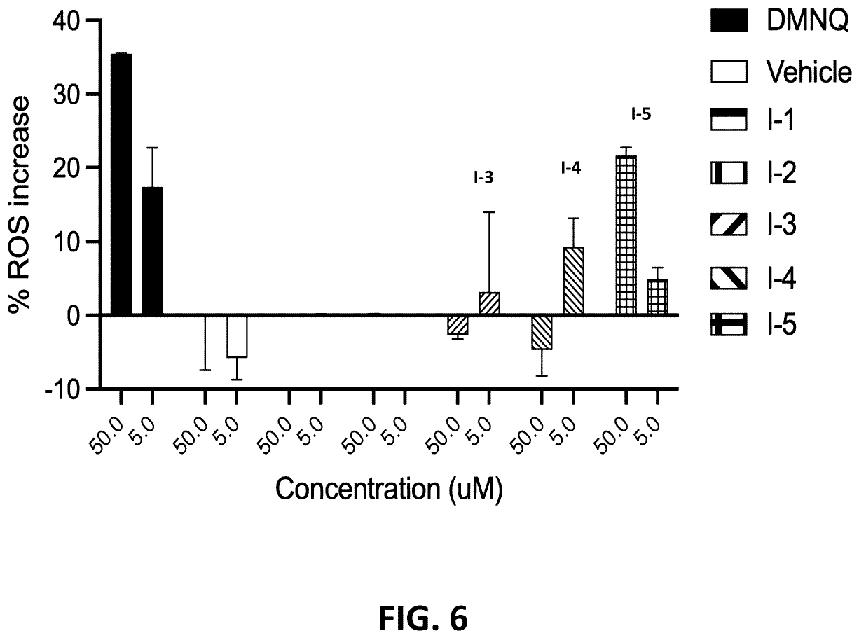

A ROS-Glo™ assay (Promega) was employed to assess the production of intracellular reactive oxygen species (ROS) for the example compounds at concentrations of either 50 or 5 μM. Referring to FIG. 6, the majority of example compounds did not induce oxidative stress in primary human follicle dermal papilla cells (hFDPCs). Elevated levels of ROS were observed exclusively in cells treated with compound I-5. Vehicle represents 0.1% DMSO and DMNQ represents 2,3-dimethoxy-1,4-naphthalenedione, a known ROS-inducer.

### Example 13: Clinical Repeat Insult Patch Test

The clinical test was conducted by ALS Pharmaceutical, adhering to established, standardized procedures for clinical testing to ensure the well-being of study subjects and the generation of reliable data. A total of 80 male and female participants, aged between 18 and 62 years, were selected for the study. The objective of the study was to evaluate the potential of the test material containing 0.2% v/v of compound I-1 (in formulation) to cause dermal irritation and/or induce sensitization following repeated patch applications. As shown in TABLE 14, none out of 59 participants reported adverse reactions.

TABLE 14
Clinical repeat insult patch test results

| Subjects Completed | Sex | Patch type | Adverse reactions |
|---|---|---|---|
| 59/80 | 11 males and | Semi-Occlusive | 0/59 (0%) |
| | 48 females | | 1 subject exhibited |
| | | | erythema, but was |
| | | | excluded from the |
| | | | study due to an |
| | | | unrelated event. |

### Example 14: Combination Therapy

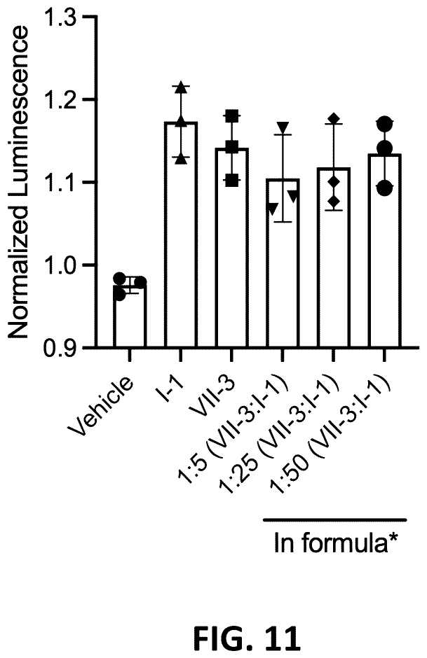

As shown in FIG. 11, compound I-1 in combination with compound VII-3 induces strong normalized relative proliferation of human follicle dermal papilla cells at various concentration ratios. The vehicle used is water. Solitary compound I-1 and compound VII-3 were initially dissolved in 1,3-Dimethyl-2-imidazolidinone (DMI), with the final concentration yielding 0.02% v/v in a mixture of DMI and water. The combinations of compound I-1 and compound VII-3 were prepared in a formula consisting of 10% dimethyl isosorbide, 15% ethanol, 15% propylene glycol, and 60% water. For the in-formula conditions, compound I-1 and compound VII-3 were added up to a maximum of 0.02% v/v. For example, a 1:5 (VII-3:I-1) ratio represents 1 part VII-3 and 5 parts I-1, combining to a maximum of 0.02% v/v in the tested formula.

### Example 15: Synthesis Method of Compound VII-3

Synthesis of compound VII-3 was carried out following Scheme 1 given below;

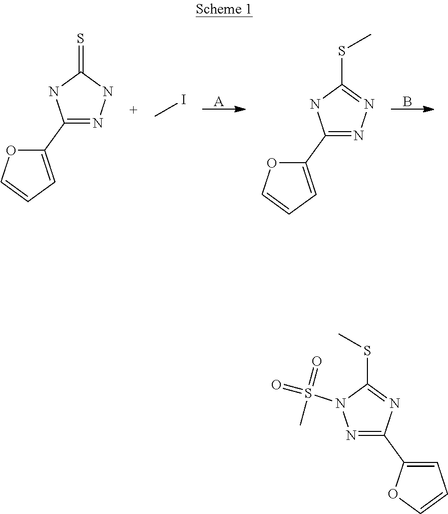

The synthesis could be performed in two different ways. As the first method, to a water solution of ethanol (EtOH) (100 ml of water and 100 ml of EtOH), NaOH was added during 20 min at room temperature. 3-(furan-2-yl)-1H-1,2,4-triazole-5(4H)-thione (20 g, 0.1 mol) was added by portions at room temperature with stirring. The mixture was stirred at room temperature for 1 h. MeI was added by drop at room temperature for 25 min and stirred the mixture for 12 h. The solution was evaporated to half the volume under reduced pressure (50° C., 20 mm), precipitate was filtered off, washed with water (3×100 ml) and dried. Final yield of the target compound was 68%.

The compound was also synthesized by the second method as follows. 3-(furan-2-yl)-5-(methylthio)-4H-1,2,4-triazole (18 g) was mixed with dry THF (125 ml) and NEt₃ was added by drop at 5° C. To a water solution of EtOH (100 ml of water and 100 ml of EtOH) NaOH was added during 20 min at room temperature. The mixture was cooled to 5° C. and MeSO₂Cl was added by drop, stirred for 1 h at 10° C. and 12 h at room temperature. 500 ml of water was added and stirred for 1 h. The precipitate was filtered, washed with water (3×80 ml), and dried. The product was purified by column chromatography (eluent MTBE/CH₂Cl₂ 50/50). Solvent was evaporated. Final yield of the target compound was 72%.

### Example 16: Synthesis Method of Compounds of Formula (I)

A general reaction scheme applicable to all library compounds is depicted in Scheme 2. The R₁ moiety was introduced via alkylation to obtain building block 3. The R₂ moiety was introduced via a sulfonyl chloride coupling to obtain the final compounds.

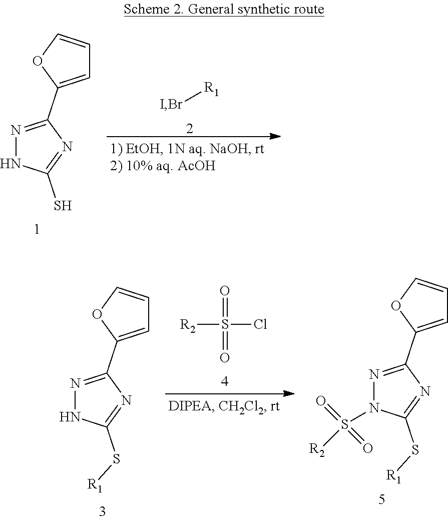

### General Procedure for Alkylation (R₁ Introduction)

Bromide or iodine 2 (1.2 equiv.) was added to a solution of 3-(furan-2-yl)-1H-1,2,4-triazole-5-thiol 1 (1.0 equiv.) in absolute ethanol (0.3 M). Aqueous sodium hydroxide (1M, 5.0 equiv.) was added and the mixture was stirred for 20 hours at room temperature. 10% aqueous acetic acid was added to neutralize the reaction mixture. The mixture was extracted with ethyl acetate (4×, 100 mL). The organic phases were combined and successively washed with saturated aqueous NaHCO₃, H₂O and brine. The organic phase was dried over Na₂SO₄ and concentrated in vacuo to obtain building block 3, which was used as such in the next step.

### General Procedure for Sulfonyl Chloride Coupling (R₂ Introduction)

Sulfonyl chloride 4 (0.23 mmol, 1.1 equiv.) and N,N-diisopropylethylamine (92 μL, 0.53 mmol, 2.5 equiv.) were added to 1 mL stock solution (0.21 mmol per mL) of building block 3 in dichloromethane. The mixture was stirred 20 hours at room temperature and concentrated in vacuo. The residue was purified by acidic preperative liquid chromatography—mass spectrometry (LC-MS) to obtain the final compound.

### Complete Reaction Schemes

The complete reaction schemes of the compounds are depicted below (Schemes 3 to 8), including the corresponding yields.

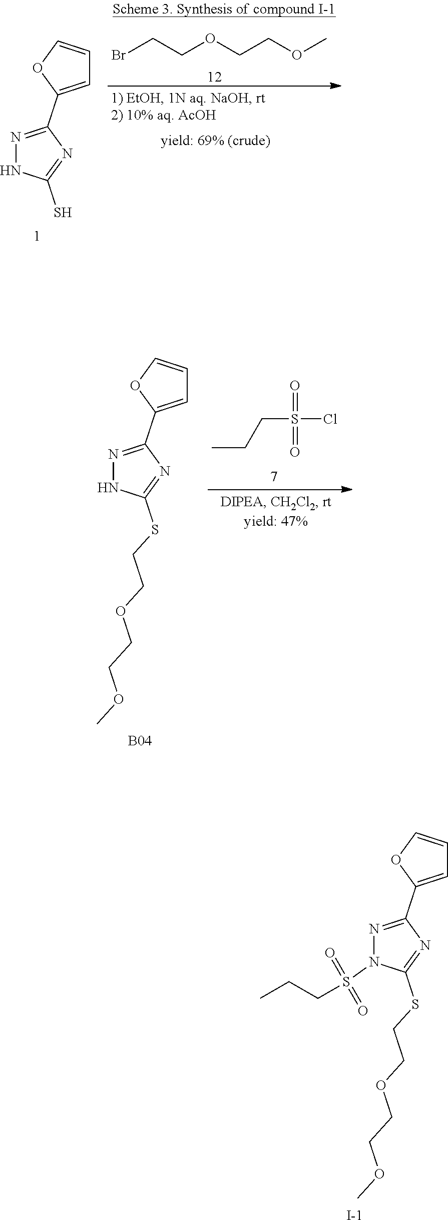

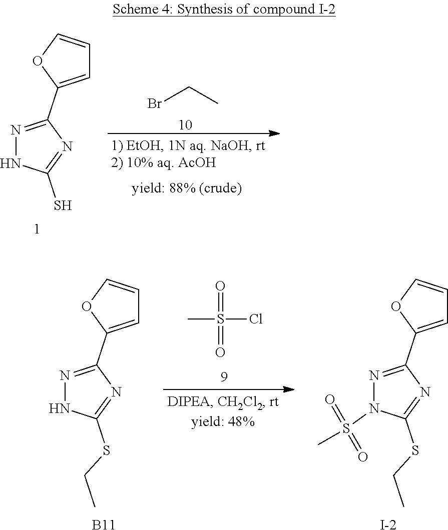

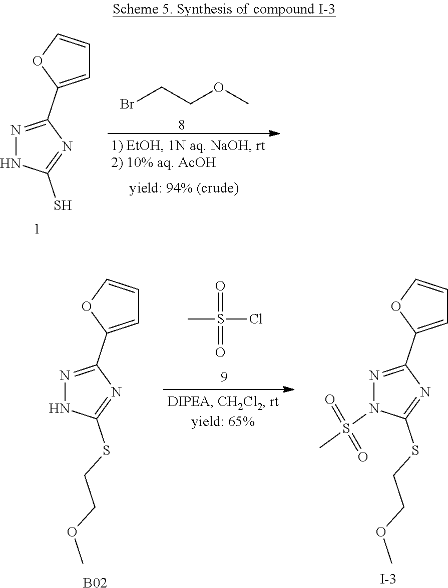

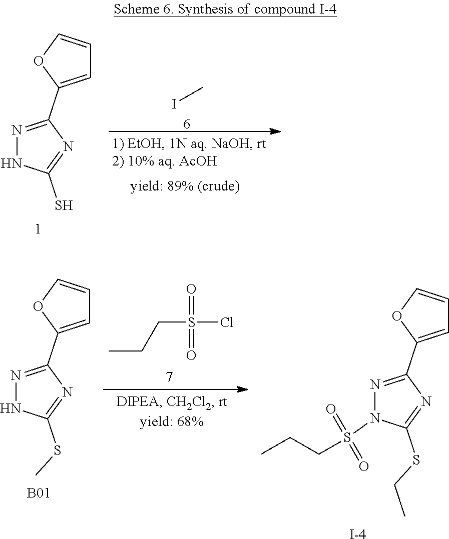

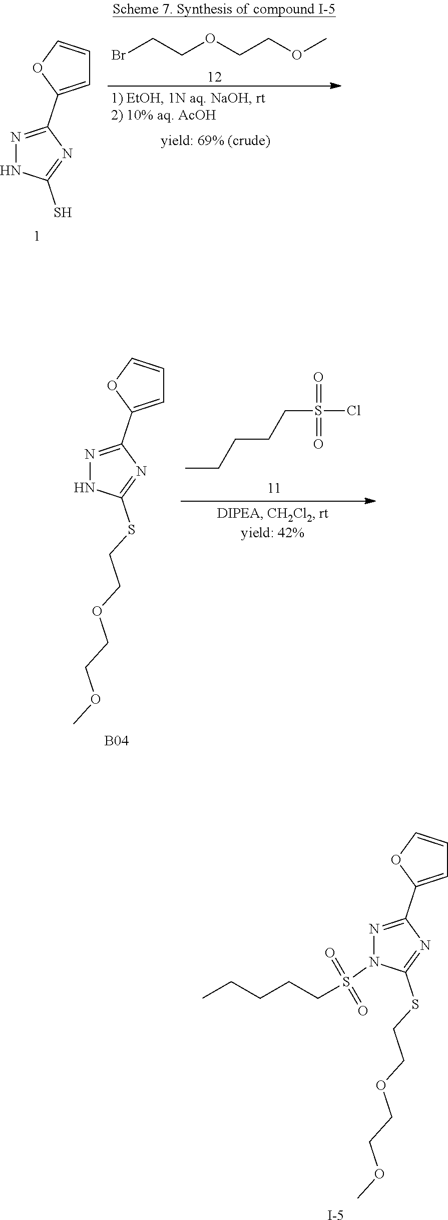

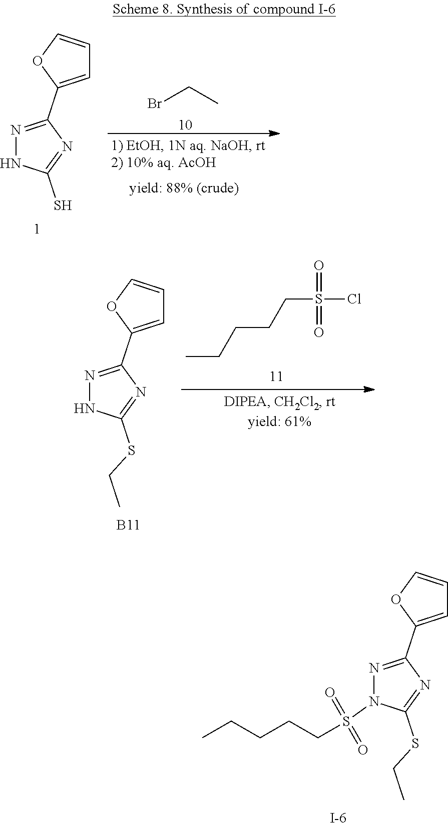

### Example 17: An Alternative Synthesis Method of Compound I-1

Scheme 9a depicts the selected and optimized intermediate reaction:

compound 1+compound 2→compound 2a.

Intermediate 2a serves as a precursor to compound 3, which is an important intermediate for the synthesis of compound I-1. Scheme 9b presents the complete reaction scheme for the synthesis of compound I-1, excluding the intermediate reactant 2a.

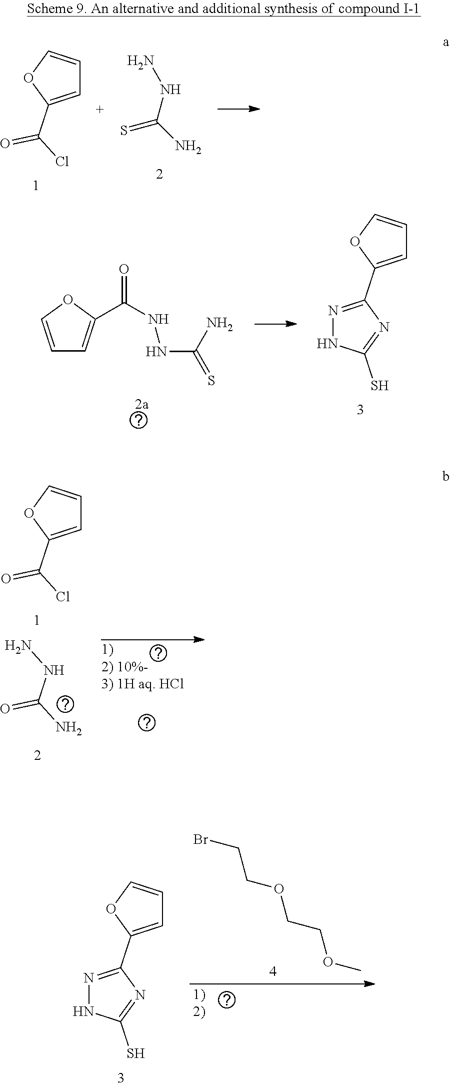

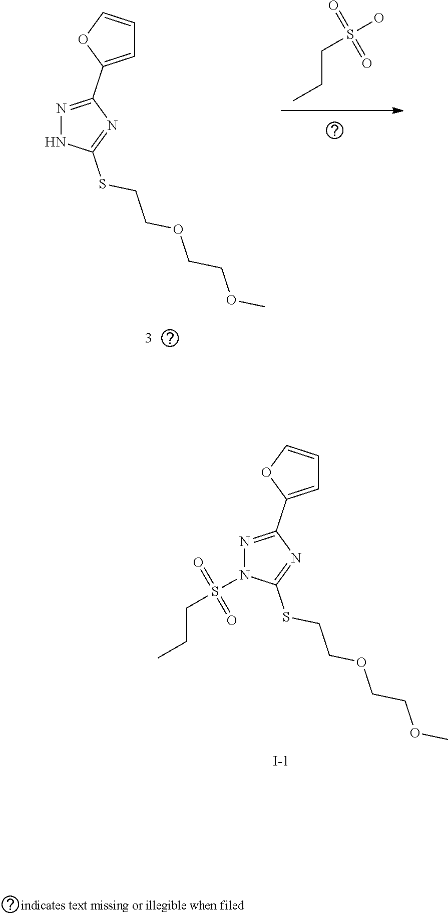

### Salt Purification of Intermediate 3-B04

To reduce color impurities in the intermediate 3-B04 in Scheme 9b, precipitation tests were conducted. A total of 290 mg of the isolated compound 3-B04 was suspended in tert-butyl methyl ether (TBME), followed by the addition of 3N HCl in cyclopentyl methyl ether (CPME). Upon adding HCl, a white precipitate formed. The solid rapidly crashed out, and the mixture turned into a clear, colorless liquid containing a pinkish solid on the glass surface. The mixture was left stirring overnight at room temperature. On the following day, the mixture was sonicated, and the resulting pinkish suspension was filtered. The solid was then thoroughly washed with TBME and dried under vacuum. A total of 201 mg of 3-B04·HCl was obtained as a pinkish solid, indicating that the purification of 3-B04 by salt precipitation rather than column chromatography should be possible in scale-up to avoid column chromatography. Further investigations into purification via salt formation are planned for future studies.

### Synthesis Optimization Conditions for Reaction: Compound 3→Compound 3-B04

A total of 5.0 g of isolated compound 3 was suspended in 75 mL of ethanol (EtOH). Subsequently, 100 mL of a 1M NaOH solution was added, which caused the solid to dissolve. The reaction was slightly exothermic, and the exothermic effect was controlled using an ice-water bath. Upon adding the base, the mixture transformed into a yellow solution. Compound 4 (Scheme 9b) was introduced at room temperature, and the mixture was stirred continuously at room temperature. The stirring process was maintained overnight. Crude product 3-B04 was obtained as a yellowish oil with a purity of 94.87%, as determined by liquid chromatography-mass spectrometry (LC-MS). The crude product was further purified using column chromatography, resulting in the removal of impurities to yield two fractions. The first fraction comprised isolated compound 3-B04 (5.18 g, 64% yield), which appeared as a slightly yellowish oil with a purity of 99.86% (the analysis was performed at 254 nm). The second fraction contained 290 mg of isolated compound 3-B04 (4% yield), which initially appeared as a yellowish-greenish oil but transitioned into a pink oil after standing for a brief period. Following purification and overnight storage of the pure material at 4-5° C., the combined fractions changed from yellowish to pink.

### Synthesis Optimization Conditions for Reaction: Compound 3-B04→Compound I-1

The product was obtained after stirring the mixture for 40 minutes at 0-2° C. An impurity with the same mass as compound I-1 was observed, likely a regioisomer of the desired product. The ice-water bath was removed, and the mixture was allowed to warm up to room temperature while stirring for 1 hour before work-up. Crude product I-1 (7.82 g) was obtained as a dark brown oil.

The purity of the crude product was analyzed by LCMS prior to column chromatography. The impurity with a retention time (Rt) of 5.15 minutes appeared to have disappeared after the work-up. However, upon removing the solvent after completion, it seemed that the starting material 3-B04 reappeared. This observation indicates that the product partially hydrolyzed (7-8% of hydrolysis) back to reactant 3-B04. Following purification by column chromatography, the isolated compound I-1 (3.0 g, 8.0 mmol, 83%) was obtained as a brown oil.

For the commercial-scale production of compound I-1, compound 3 in Scheme 9 (which is also compound 1 in Scheme 3) was synthesized in larger quantities to facilitate the scale-up process. The reaction of [compound 1+compound 2→compound 2a] in Scheme 9A was ultimately selected to be performed in a 10:1 pyridine solution, as this approach yielded high conversion, purity, and processability.

TABLE 15
Experimental parameters and purity results for optimization of Reaction:
compound 1 + compound 2 → compound 2a in Scheme 9A.

| | | Volume | | | ROCl | |
|---|---|---|---|---|---|---|
| ID | Solvent | Ratio | Base | Yield | Eq.* | Purity |
| Exp a | DCM | 10 | None | 64% | 1.1 | Almost Pure |
| Exp b | Pyridine | 10 | None | 126% | 1.1 | Almost Pure |
| Exp c | DCM | 20 | 1.1 eq. | Not | 1.1 | Many impurities |
| | | | Pyridine | reported | | |
| Exp d1 | DCM | 20 | NEt₃ | Incomplete | 1.0 | Many impurities |
| Exp d2 | DCM | 20 | DIPEA | Incomplete | 1.0 | Many impurities |
| Exp d3 | DCM | 20 | DBU | Incomplete | 1.0 | Many impurities |
| Exp d4 | DCM | 20 | K₂CO₃ | Incomplete | 1.0 | Many impurities |
| Exp e | Toluene | 20 | None | 81% | 1.1 | Almost Pure |
| Exp f1 | DCM | 20 | None | 79% | 1.1 | Almost Pure |
| Exp f2 | DCM | 20 | None | Not | 1.5 | Some impurities |
| | | | | reported | | |
| Exp g | 1-BuOH | 20 | None | Not | 1.1 | Many impurities |
| | | | | reported | | |
| Exp h | Pyridine | 10 | None | 131% | 1.1 | Almost Pure |

*ROCl is the equivalent of Acid Chloride reagent. In this case the ROCl is (1) in the reaction synthesis mechanism (furoyl chloride).

Exp a: A total of 500 mg of thiosemicarbazide was suspended in 10 vol of dichloromethane (DCM). The term “vol” represents volume equivalents to limiting reagent. In this case, because 500 mg of thiosemicarbazide, 10 vol would be 5 mL (10×500 mg). The mixture was cooled down to 0-2° C. with an ice/water bath, and 1 eq of furoyl chloride was added, over 5 min. The suspension became slightly pinkish. The mixture was left stirring overnight at room temperature to produce a white solid, which was triturated in DCM and filtered off. After washing with excess DCM and drying, crude product 2a·HCl (780 mg, 5.49 mmol, 64%) was obtained as a white solid.

Exp b: A total of 500 mg of thiosemicarbazide were suspended in 10 vol of pyridine. The mixture was cooled down to 0-2° C. with an ice/water bath, and 1 eq of furoyl chloride was added, over 5 min. The mixture became yellow. The mixture was stirred overnight at room temperature, and the pyridine was removed in vacuo. The resulting yellow residue was triturated in DCM. The solid was filtered off, washed with plenty DCM, and dried. Crude product 2a (1.26 g, 5.49 mmol max., 126%) was obtained as a white solid.

Exp c: The reaction was performed on a 0.5 g scale in DCM (20 vol), with 1.1 eq of pyridine. The addition of furoyl chloride was performed at 0-2° C., and the mixture was left warming up to room temperature overnight. The mixture became yellowish upon adding the furoyl chloride. The following day, the mixture was a fine yellow suspension. Conversion was rather incomplete and, surprisingly, quite a few impurities were formed. Still, the pyridine was removed in vacuo and the resulting residue was triturated in DCM. After filtration, washing and drying of the yellowish solid. A base screening was performed to see if fewer impurities could be formed.

Exp d1 to d4: Each reaction was performed on a 0.5 g scale, in 20 vol of DCM, with 1.1 eq of the appropriate base including triethylamine (NEt₃), N,N-diisopropylethylamine (DIPEA), 1,8-Diazabicyclo[5.4.0]undec-7-ene (DBU), K₂CO₃. The addition of 1.0 eq of furoyl chloride was performed at 0-2° C. For each reaction, many impurities were observed (though K₂CO₃) gave the cleanest conversion. All the reaction mixtures were suspensions after stirring overnight at room temperature. In all cases, the reaction was not complete. Therefore a solvent screening without base was performed. Each reaction was performed in 20 vol of the appropriate solvent, without base. The addition of furoyl chloride was performed at 0-2° C., and the mixture were left stirring overnight, while slowly warming up to room temperature.

Exp e: The reaction was performed in 20 vol of toluene. The addition of furoyl chloride (1.1 eq) was performed at 0-2° C. After stirring the mixture overnight at room temperature, it became a very thick suspension, very difficult to stir properly. Two main impurities were formed, in small amounts. The mixture was filtered off and the solid washed with sufficient DCM and dried in vacuo. Crude product 2a (3.32 g, 22.1 mmol, 81%) was obtained as a white solid.

Exp f1-2: DCM was tested again, with 1.1 eq of acid chloride (Exp f1) and with 1.5 eq of acid chloride (Exp f2). The excess of acid chloride seemed to have caused the formation of the minor impurities. The starting material was completely consumed.

Exp g: High boiling point alcohol 1-BuOH was also tested. As expected, the solvent reacted with the acid chloride causing the formation of furoyl butanoate, in high amounts. While DCM and toluene both proved to be good solvents for the reaction. Due to the low yields observed, it was determined to optimize the reaction in pyridine, by reducing the volume equivalents of pyridine and maintaining temperature below 5° C. during the acid chloride addition:

Exp h: The reaction was performed in 10 vol of pyridine, with 1.1 eq of acid chloride. The addition was performed, over 20-25 min, while the temperature was kept below 5° C. After complete addition, the mixture became a completely clear yellow solution. After 15-20 min at lower temperature, a precipitate started to form. The mixture was then stirred overnight while slowly warming up to room temperature. The mixture was then treated with DCM (100 mL, 20 vol) and left stirring at room temperature for 20 min. The mixture was filtered off and the solid washed with plenty of DCM. Crude product 2a (13.35 g, 55.0 mmol max., 131%) was obtained as a white/very slightly yellowish solid. A second repeat at this scale and conditions was performed. Yielding similar results.

To prepare compound I-1 in commercial quantities, compound 2a in Scheme 9a was synthesized in larger quantities for scale-up. During this process, compound 2a generated in pyridine demonstrated superior yield and purity during cyclization compared to that produced in toluene. Trituration and recrystallization of compound 3 in water were chosen for scale-up due to the optimal combination of cost, yield, and purity factors.

TABLE 16
Experimental parameters and purity results for optimization of
Reaction: compound 2a → compound 3 in Scheme 9.

| ID | Solvent | Scale | Work-up | Yield | Purity | Color |
|---|---|---|---|---|---|---|
| Exp aa | Toluene | 3.32 g | DCM-Wash/ | 53% | 96.2% | Off-white |
| | | | Filtration | | | |
| Exp bb | Pyridine | 5.0 g | DCM-Wash/ | 60% | 98.5% | Tan |
| | | | Filtration | | | |
| Exp cc | Pyridine | 5.0 g | In-vacuo drying | 79% | 97.8% | Yellow |

Exp aa: A total of 40 mL of a 10% KOH solution was added to 3.32 g of compound 2a, resulting in an orange solution that was refluxed for 3 hours. LCMS analysis confirmed complete conversion without the formation of impurities. The mixture was then cooled to 0-2° C. and acidified with 2M HCl to achieve a pH of 1-2. The mixture was stirred at 0-2° C. for 30 minutes before being filtered. The solid was washed with an excess of water and dried under vacuum. Isolated compound 3 (1.6 g, 9.6 mmol, 53% yield) was obtained as a slightly off-white solid with a purity of 96.2%. It is worth noting that reactant 2, present in the crude product 2a, could be entirely washed away during the isolation of compound 3.

Exp bb: The reaction mixture was treated with DCM and the solid 2a was isolated by filtration.

Exp cc: The reaction mixture was concentrated in vacuo to remove the pyridine.

Both materials from exp bb and cc were then ring-closed, under the same conditions.

In order to obtain the most pure and colorless compound 3 possible, various trituration and recrystallization methods were tested. A total of 100 mg of crude, tan-colored compound 3 was suspended in microwave vials. Either 10 or 20 volumes of the appropriate solvent were added, followed by refluxing the mixtures, cooling them down to room temperature, and then filtering and drying. It was determined that purifying crude compound 3 in water was the most suitable approach, as this solvent is the most environmentally friendly, safe, and cost-effective option.

TABLE 17
Experimental parameters for purity and color
results of compound 3 in Scheme 9.

| | | | |
|---|---|---|---|
| Solvent | Recovery | Purity (Solid) | Color |

| | | | | |
|---|---|---|---|---|
| H₂O | 104 | mg | 93.5% | White |
| ACN | 102 | mg | 85.4% | White |
| EtOH | 90 | mg | 98.2% | White |
| H₂O/ACN (1:1) | 68 | mg | 97.4% | White |
| H₂O/EtOH (1:1 | 85 | mg | 97.1% | White |

### Example 18: Evaluation of Stability of Compound I-1

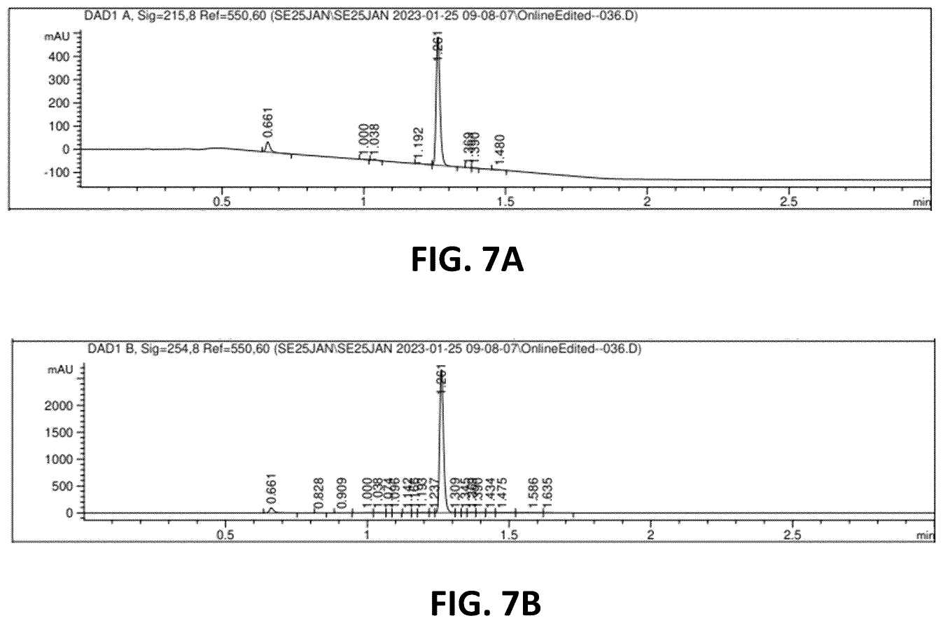

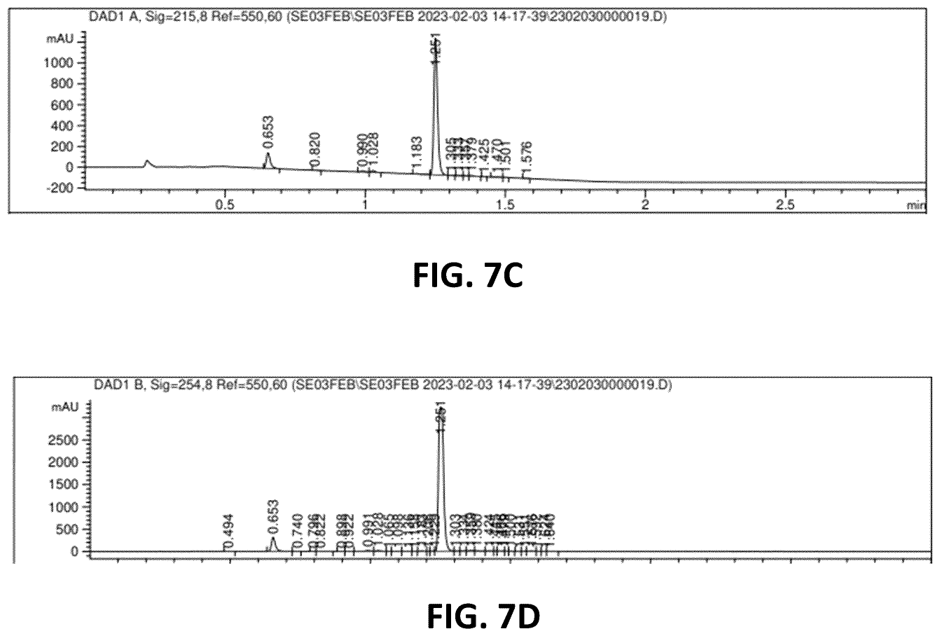

A sample was analyzed for stability using LCMS. In both cases, the peak at 1.25 min was determined to be product I-1, while the impurity at 0.66 min was identified as the hydrolysis product 3-B04, as referenced in Scheme 9b. After analysis, the sample was allowed to stand in open air overnight. FIG. 7A displays the UV-traces of the freshly synthesized material post-workup at 215 nm and FIG. 7B exhibits the UV-traces of the freshly synthesized material post-workup at 254 nm. FIG. 7C presents the UV-traces of the material after 24 hours of open-air exposure at 215 nm, and FIG. 7D displays the UV-traces of the material after 24 hours of open-air exposure at 254 nm. The purity of the compound in FIG. 7B was analyzed to be 95.3% (254 nm), and in FIG. 7D to be 90.61% (254 nm) after standing overnight in air. In conclusion, it was observed that compound I-1 hydrolyzed upon storage in open air at room temperature. The compound gradually decomposed into compound 3-B04 and propane sulfonic acid.

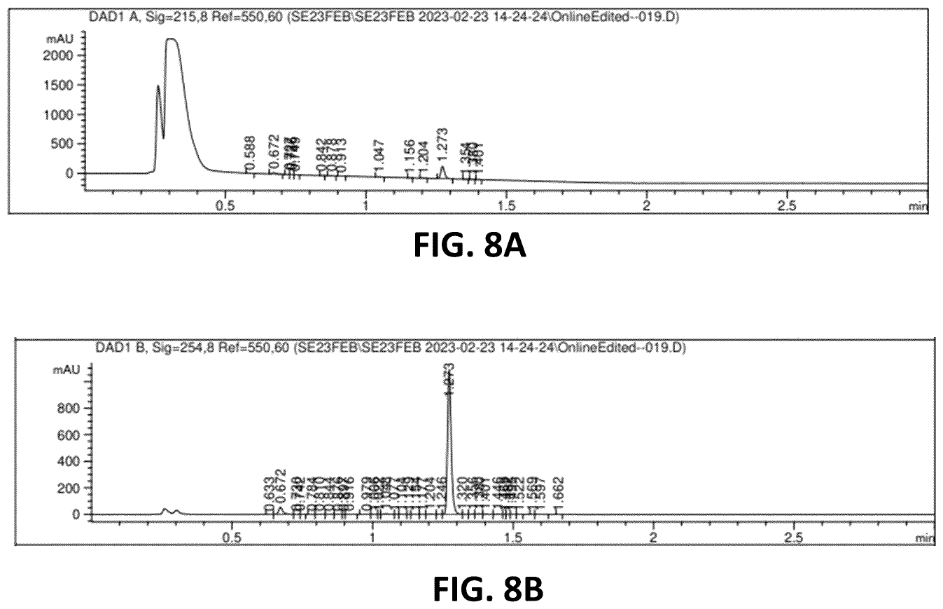

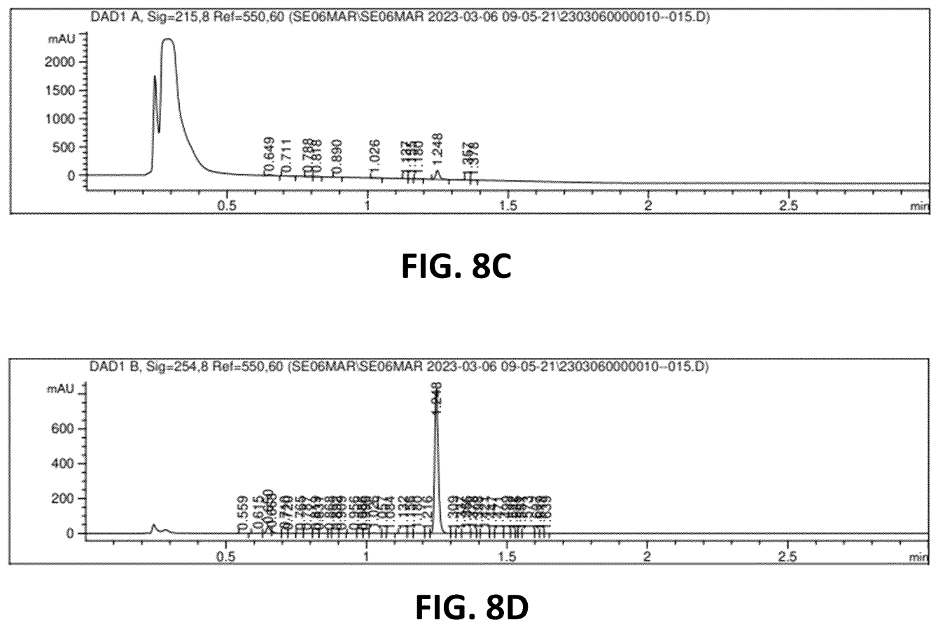

A sample containing compound I-1 was prepared in 1,3-Dimethyl-2-imidazolidinone (DMI), sealed, and stored at room temperature for 7 days. Another sample containing compound I-1 was prepared in DMI and left open for 8 days. The purity of both samples was assessed using LCMS. FIG. 8A displays the UV-traces of the closed-air experiment at 215 nm, and FIG. 8B exhibits the UV-traces of the closed-air experiment at 254 nm. FIG. 8C presents the UV-traces of the open-air experiment at 215 nm, and FIG. 8D shows the UV-traces of the open-air experiment at 254 nm. The purity in FIG. 8B was determined to be 92.9%, and the purity in FIG. 8D was also found to be 92.9%. Compound I-1 appeared to be stable in DMI, even when the solution was exposed to air.

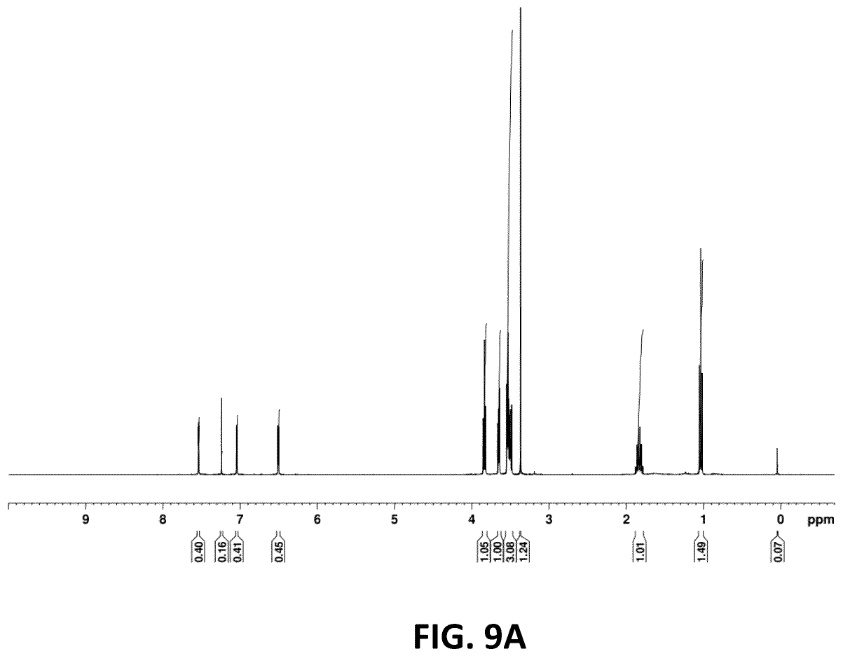

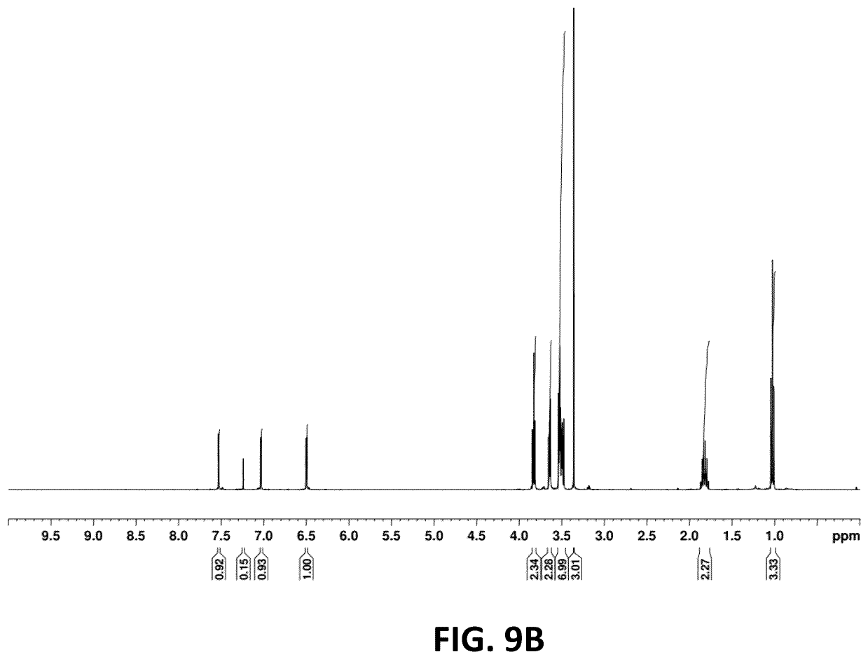

Thermal degradation of compound I-1 was evaluated at 160° C. and 185° C. Pure compound I-1 (1.2 g), in the form of a brown oil, was subjected to distillation for 30 minutes at 160° C. When no product was observed, distillation was attempted at 185° C., albeit unsuccessfully. NMR analysis indicated that the compound was slightly decomposing at this temperature, as shown in FIG. 9A and FIG. 9B

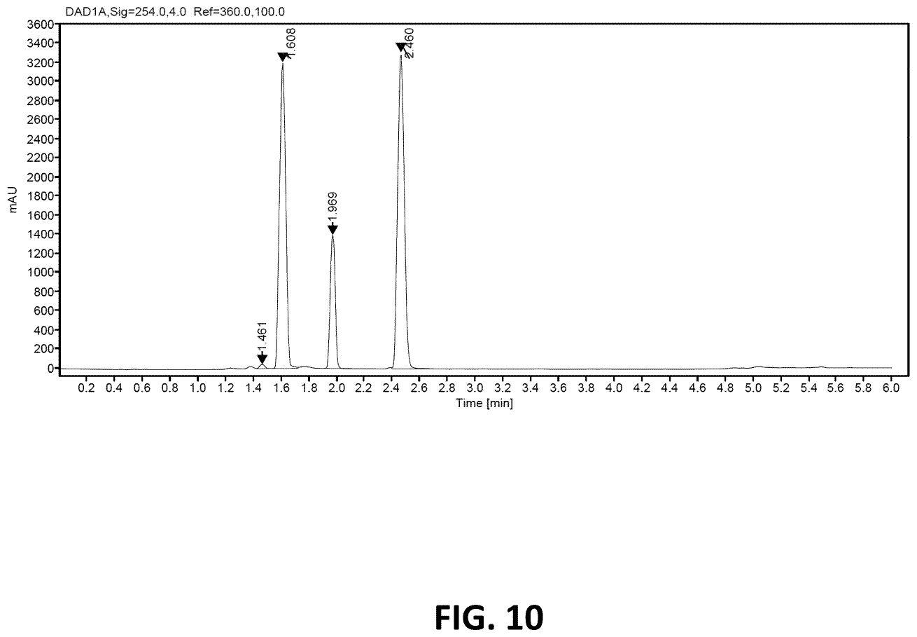

Example of composition (TABLE 2-TABLE 11) preserves the structure and inhibits the hydrolysis of compound I-1, even after storage of 8 months at room temperature. HPLC-UV analysis reveals minimal hydrolysis of compound I-1 (retention time of 2.46 minutes) into the major hydrolysis product (retention time of 1.461 minutes) after storage of 8-months at room temperature, as shown in FIG. 10.

While preferred embodiments of the present invention have been shown and described herein, it will be obvious to those skilled in the art that such embodiments are provided by way of example only. Numerous variations, changes, and substitutions will now occur to those skilled in the art without departing from the invention. It may be understood that various alternatives to the embodiments of the invention described herein may be employed in practicing the invention. It is intended that the following claims define the scope of the invention and that methods and structures within the scope of these claims and their equivalents be covered thereby.

1. A composition comprising a compound having a structure of Formula (I): 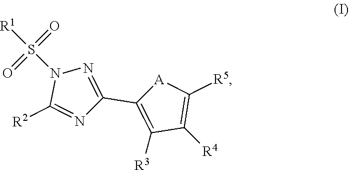
or a salt thereof,
wherein:
R¹ is selected from hydrogen, —OH, C₁₋₆ alkyl, and C₁₋₆ haloalkyl;
R² is selected from hydrogen, —OH, C₁₋₆ alkoxy, C₁₋₆ haloalkoxy, —SH, —S-alkyl, —S-haloalkyl, and —S—(CH₂CH₂—O)ₙ—R^{a}; wherein
n is an integer from 0 to 6, and
R^{a} is C₁₋₆ alkyl or C₁₋₆ haloalkyl;
A is selected from NH, O, and S; and
R³, R⁴, and R⁵ are independently selected from hydrogen, —OH, C₁₋₆ alkyl, and C₁₋₆ haloalkyl.

2. The composition of claim 1, wherein R¹ is C₁₋₆ alkyl.

3. The composition of claim 1, wherein R¹ is selected from —CH₃, —(CH₂)₂CH₃, and —(CH₂)₄CH₃.

4. The composition of claim 1, wherein R² is —S-alkyl.

5. The composition of claim 4, wherein R² is —S—CH₃ or —S—CH₂CH₃.

6. (canceled)

7. The composition of claim 1, wherein R² is —S—(CH₂CH₂—O)ₙ—R^{a}.

8. The composition of claim 7, wherein n is 1 or 2.

9. The composition of claim 7 or 8, wherein R^{a} is C₁₋₆ alkyl.

10. The composition of claim 9, wherein R^{a} is —CH₃.

11. The composition of claim 1, wherein A is O.

12. The composition of claim 1, wherein R³ is hydrogen.

13. The composition of claim 1, wherein R⁴ is hydrogen.

14. The composition of claim 1, wherein R⁵ is hydrogen.

15. The composition of claim 1, wherein the compound of Formula (I) is selected from: 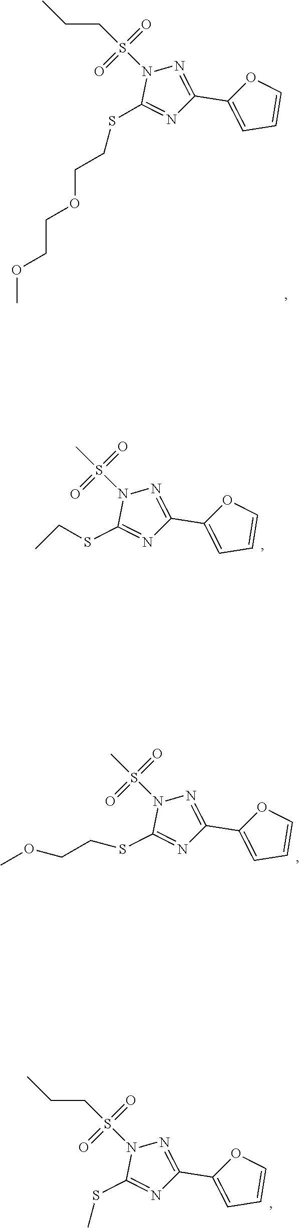 [SMILES: CCCS(=O)(=O)n1nc(-c2ccco2)nc1SCCOCCOC ; CCSc1nc(-c2ccco2)nn1S(C)(=O)=O ; COCCSc1nc(-c2ccco2)nn1S(C)(=O)=O ; CCCS(=O)(=O)n1nc(-c2ccco2)nc1SC] 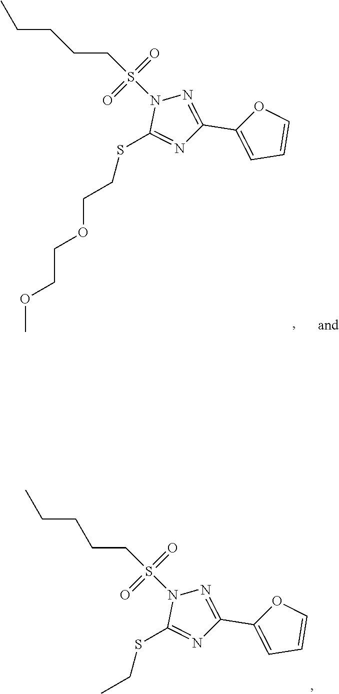 [SMILES: CCCCCS(=O)(=O)n1nc(-c2ccco2)nc1SCCOCCOC ; CCCCCS(=O)(=O)n1nc(-c2ccco2)nc1SCC]
or a salt thereof.

16. The composition of claim 15, wherein the compound of Formula (I) is 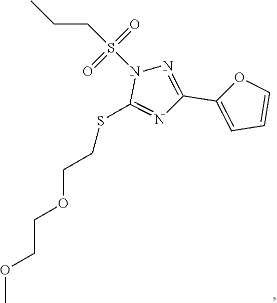 [SMILES: CCCS(=O)(=O)n1nc(-c2ccco2)nc1SCCOCCOC]
or a salt thereof.

17. The composition of claim 1, comprising the compound in a weight % ranging from about 0.0001% to about 10.0% relative to a total weight of the composition.

18-29. (canceled)

30. The composition of claim 1, wherein the composition maintains chemical stability of the compound of Formula (I) during storage at room temperature for at least about 4, 6, or 8 months by preserving the chemical structure and/or prevents hydrolysis/degradation of the compound of Formula (I).

31. (canceled)

32. The composition of claim 1, wherein the composition is used for treating hair loss or hair thinning of a subject in need thereof, wherein the hair loss or hair thinning of the subject is selected from androgenic alopecia, alopecia areata, androgenetic alopecia, gynecologic alopecia, postpartum alopecia, seborrheic alopecia, non-rigid alopecia, senile alopecia, chemotherapy-induced alopecia, radiation-induced alopecia, male-pattern baldness, female-pattern baldness, cicatricial alopecia, alopecia areata telogen effluvium, traction alopecia, anagen effluvium, and a combination thereof.

33-35. (canceled)

36. The composition of claim 1, wherein the composition is applied to a scalp hair, an eyelash hair, an eyebrow hair, a facial hair, or a combination thereof.

37-79. (canceled)

80. A composition comprising the compound of claim 15 and 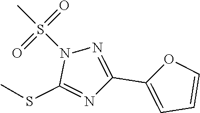 [SMILES: CSc1nc(-c2ccco2)nn1S(C)(=O)=O]
or a salt thereof.
